(19) United States
(12) Patent Application Publication
(10) Pub. No.: US 2025/0144125 A1
(43) Pub. Date: May 8, 2025

(54) 6-SUBSTITUTED- AND 6,7-DISUBSTITUTED-7-DEAZAPURINE RIBONUCLEOSIDE ANALOGUES

(71) Applicant: KATHOLIEKE UNIVERSITEIT LEUVEN, Leuven (BE)

(72) Inventors: Piet HERDEWIJN, Leuven (BE); Zihua ZHENG, Hubei (CN); Elisabetta GROAZ, Leuven (BE)

(21) Appl. No.: 18/684,405

(22) PCT Filed: Aug. 18, 2022
(86) PCT No.: PCT/EP2022/073063

§ 371 Date: Feb. 16, 2024

(30) Foreign Application Priority Data
Aug. 18, 2021 (EP) 21191817.2
Aug. 18, 2021 (EP) 21191922.0
Feb. 1, 2022 (EP) 22154636.9

(51) Int. Cl. A61K 31/7064; A61P 31/14; A61P 31/16; C07H 19/14

(52) U.S. Cl. CPC A61K 31/7064; A61P 31/14; A61P 31/16; C07H 19/14

(57) ABSTRACT

The present invention relates to a compound with general structure (I) or (II) or a pharmaceutically acceptable salt or prodrug thereof

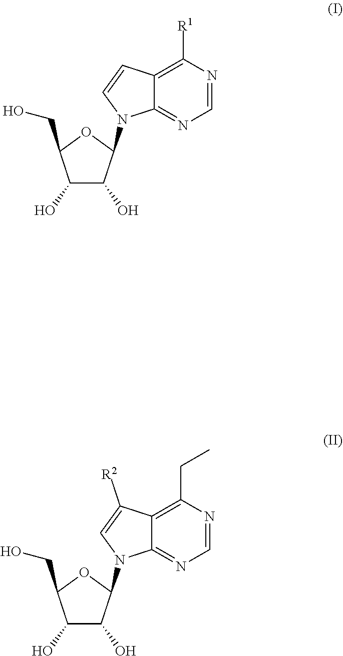

wherein R¹ and R² have the same meaning as that defined in the claims and the description. The present invention also relates to pharmaceutical compositions comprising such compounds and to uses of such compounds and compositions for the treatment or prevention of viral infections, more in particular infections caused by RNA virus.

### FIELD OF THE INVENTION

The invention relates to the treatment and prevention of viral infections, in particular RNA viral infections.

The invention relates to the synthesis of novel 6-substituted- and 6,7-disubstituted-7-deazapurine ribonucleoside analogues.

### BACKGROUND OF THE INVENTION

Tubercidin, a natural product¹, is an analogue of adenosine with the N-7 replaced by C—H (purine numbering is used herein) and exhibits potent antitumor and antiviral activity.² Upon phosphorylation in cells, it is converted to the corresponding triphosphate, which is a substrate for polymerases and incorporated into DNA and RNA.³ Numerous tubercidin analogues were studied as potential cytostatic or antiviral agents, such as 6-substituted⁴⁻⁶, 7-substituted⁷, 6,7-disubstituted 7-deazaadenosine analogues⁸, sugar-modified 7-deazaadnosine analogues⁹ and sugar-modified derivatives of 7-(het)aryl 7-deazaadenosine^{10,11}. According to structure-activity studies, 6- or 7-substituted 7-deazaadenosine derivatives with small or 5-membered hetaryl substituents showed potent antitumor activity against a broad panel of cancer cell lines¹², while sugar-modified analogues normally caused less cytostatic effect.

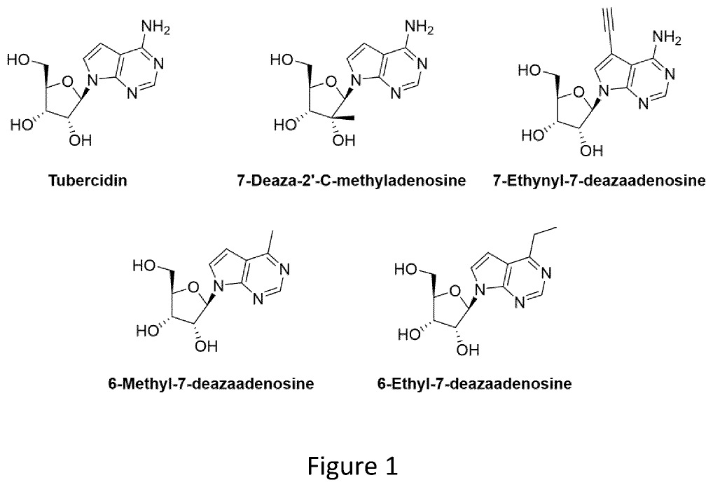

Although tubercidin has no clinical use as an antiviral agent because of its cytotoxicity, it is still an attractive lead compound to develop selective antiviral agents via chemical modifications. 7-Deaza-2′-C-methyladenosine (FIG. 1) was identified as a potent inhibitor of HCV RNA replication (EC₅₀=0.25 μM) with no significant cytotoxicity and 7-fluoro-7-deaza-2′-C-methyladenosine displayed an EC so of 0.07 μM against HCV.⁹ When the 6-amino group is replaced by a nonpolar methyl group, 6-methyl-7-deazaadenosine is obtained. This compound showed inhibitory effect against replication of poliovirus (PV, IC₅₀=11 nM) and dengue virus (DENY, IC₅₀=62 nM).⁶ In addition, 6-methyl-7-deazaadenosine was evaluated against HCV, showing an EC₅₀ of 0.02 μM with significant cytotoxicity (CC₅₀=0.23 μM).⁸ Recently, Hocek and co-workers identified the activity of 7-ethynyl-7-deazaadenosine against ZIKA and SARS-CoV-2.¹³ Although submicromolar activity was observed, the compound was proven to be nonselective.

Among a series of 6-substituted derivatives, 6-ethyl-7-deazaadenosine displayed micromolar cytostatic activity against multiple tumor cell lines.⁴ Normally, palladium catalysed cross-coupling was used to introduced different substituents at the 6-position. In our previous work, 6-alkyl- and aryl-7-deazaadenosine derivatives were synthesized via Fe/Cu co-catalysed coupling reaction.⁵ Their antiviral activity against various RNA viruses was further studied.

### SUMMARY OF THE INVENTION

The present invention describes the synthesis of 6-substituted-7-deazaadenosine and 6,7-disubstituted-7-deazaadenosine analogues. These 7-deazaadenosine derivatives were evaluated for their antiviral activity and cytotoxicity against five representative RNA viruses, including MERS-CoV, measles virus, tacaribe virus, yellow fever virus and influenza A virus. In addition, 6-substituted- and 6,7-disubstituted-7-deazaadenosine analogues were evaluated against human norovirus. 6-Cyclopropylethynyl analogue 3.1c performed selective submicromolar antiviral activity against measles virus (EC₅₀=0.97 μM, SI₅₀>100), tacaribe virus (EC₅₀=0.43 μM, SI₅₀>230) and human norovirus (EC₅₀=0.13 μM, SI₅₀=139). 7-Deazaadenosine analogues with a nonterminal ethynyl substituent at the 6-position according to the invention displayed potent anti-human norovirus activity, in particular compounds 3.1d, 3.1f and 3.1h with low nanomolar activity and high selectivity. For example, 6-(1-ethoxyvinyl)-7-deazaadenosine (3.1b) exhibited an EC₅₀ of 0.007 μM and a selectivity index (SI₅₀) of 8846. Among 6,7-disubstituted derivatives, 7-ethynyl/fluoro-6-ethyl-7-deazaadenosine analogues (3.11 and 3.1r) showed submicromolar activity (EC₅₀=0.34 μM) with high selectivity.

The present invention discloses two types of 7-deazaadenosine analogues, including 6-substituted derivatives and 7-substituted 6-ethyl-7-deazaadenosine derivatives and investigated their activity against various RNA viruses.

The synthesis of two series of 7-deazaadenosine analogues, 6-substituted and 6,7-disubstituted analogues, and evaluation of their antiviral activity against various RNA viruses (MERS-CoV, measles virus, tacaribe virus, yellow fever virus and influenza A virus; human norovirus) is described herein. 6-Cyclopropylethynyl analogue 3.1c performed broad-spectrum antiviral activity without significant cytotoxicity. Furthermore, 6-substituted-7-deazaadenosine analogues according to the invention exhibited remarkable activity against human norovirus, dengue virus (DENY), yellow fever virus (YFV), Middle East respiratory syndrome coronavirus (MERS-CoV), measles virus, and tacaribe virus, as well as very high activity and high selectivity against influenza A (H1N1, H3N2, and H5N1) and influenza B viruses.

According to a first aspect of the present invention a compound with general structure (I) or (II), a pharmaceutically acceptable salt or prodrug thereof is provided,

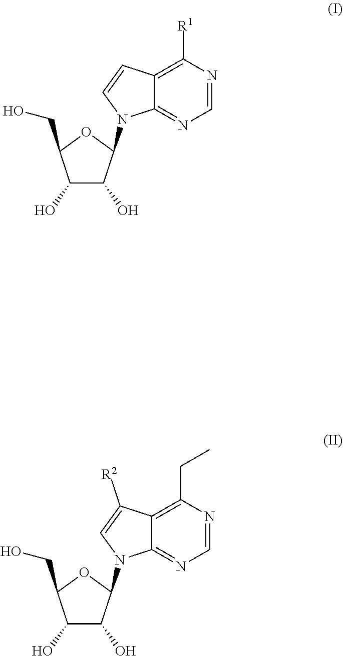

wherein R¹ is selected from the group consisting of:
vinyl, vinylC₁₋₆alkyl or C₁₋₆alkylene-vinyl, wherein the vinyl, vinylC₁₋₆alkyl or C₁₋₆alkylene-vinyl is optionally substituted with OC₁₋₆alkyl, OH, halogen, amino, dimethylamino or C₆₋₁₀aryl,
ethynyl or an ethynyl alkyl, wherein the alkyl group is a straight or branched C1 to C6 chain,
ethynyl cycloalkyl, wherein the cycloalkyl is a C3-C7 cycloalkyl, and
ethynyl benzene, wherein the benzene ring is optionally substituted with an halogen;
R² is selected from the group consisting of:
   a C2-C6 chain comprising a vinyl or ethynyl group, the C2-C6 chain optionally substituted with a C3-C6 cycloalkyl, or the C2-C6 chain optionally substituted with a benzene ring wherein the benzene ring is optionally substituted with a halogen, OH or CH₃, and
   a halogen.

According to a second aspect, the present invention also encompasses a pharmaceutical composition comprising:
a compound according to the first aspect, or a pharmaceutically acceptable salt, and
a pharmaceutical acceptable carrier.

According to a third aspect, the present invention also encompasses a compound according to the first aspect of the invention or a pharmaceutical composition according to the second aspect of the invention, for use as a medicament.

According to a fourth aspect, the present invention also encompasses a compound according to the first aspect of the invention or a pharmaceutical composition according to the second aspect of the present invention, for use in the treatment or prevention of vial infections.

Preferred statements (features) and embodiments of the compounds and processes of this invention are now set forth. Each statements and embodiments of the invention so defined may be combined with any other statement and/or embodiments unless clearly indicated to the contrary. In particular, any feature indicated as being preferred or advantageous may be combined with any other feature or features indicated as being preferred or advantageous.

The invention is further summarized in the following statements:
1. A compound with general structure (I) or (II), or a pharmaceutically acceptable salt or prod rug thereof

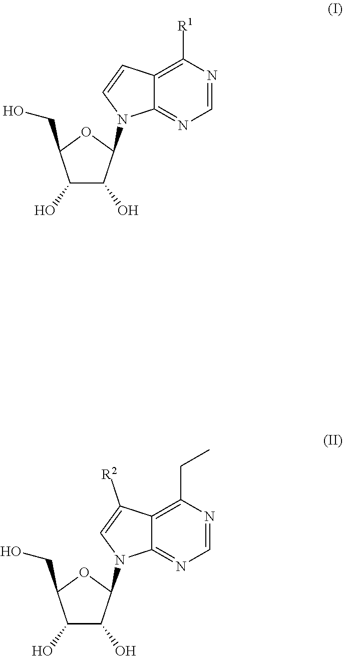

wherein R¹ is selected from the group consisting of:
   vinyl, vinylC₁₋₆alkyl or C₁₋₆alkylene-vinyl, wherein the vinyl, vinylC₁₋₆alkyl or C₁₋₆alkylene-vinyl is optionally substituted with OC₁₋₆alkyl, OH, halogen, amino, dimethylamino or C₆₋₁₀aryl,
   ethynyl or an ethynyl alkyl, wherein the alkyl group is a straight or branched C1 to C6 chain,
ethynyl cycloalkyl, wherein the cycloalkyl is a C3-C7 cycloalkyl,
and
   ethynyl benzene, wherein the benzene ring is optionally substituted with an halogen;
R² is selected from the group consisting of:
   a C2-C6 chain comprising a vinyl or ethynyl group, the C2-C6 chain optionally substituted with a C3-C6 cycloalkyl, or the C2-C6 chain optionally substituted with a benzene ring wherein the benzene ring is optionally substituted with a halogen, OH or CH₃, and
   a halogen.
2. The compound or a pharmaceutically acceptable salt or prodrug thereof according to statement 1, wherein R¹ is selected from the group consisting of: vinyl, vinylC₁₋₄alkyl, C₁₋₄alkylene-vinyl, ethynylC₁₋₄alkyl, ethynylC₃₋₅cycloalkyl, and ethynyl benzene.
3. The compound or a pharmaceutically acceptable salt or prodrug thereof according to any one of statements 1 or 2, wherein R¹ is selected from the group consisting of:
   vinyl, prop-1-enyl, allyl, 3-methylbut-1-enyl, but-1-enyl, but-2-enyl, but-3-enyl, pent-1-enyl, pent-2-enyl, pent-3-enyl, pent-4-enyl, hex-1-enyl, hex-2-enyl, hex-3-enyl, hex-4-enyl, hex-5-enyl, 4-methylpent-1-enyl, 4-methylpent-2-enyl, 3-methylhept-1-enyl, 2-methylhept-1-enyl, wherein said vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct-1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl is optionally substituted with OC₁₋₆alkyl, OH, halogen, amino, dimethylamino or C₆₋₁₀aryl,
   ethynyl, prop-1-ynyl, buty-1-ynyl, pent-1-ynyl, 3-methylbut-1-ynyl, hex-1-ynyl, 4-methylpent-1-ynyl, 3-methylpent-1-ynyl, hept-1-ynyl, 5-methylhex-1-ynyl, 4-methylhex-1-ynyl, 3-methylhex-1-ynyl, 4,4-dimethylpent-1-ynyl, oct-1-ynyl, 6-methylhept-1-ynyl, 5-methylhpet-1-ynyl, 4-methylhpet-1-ynyl, 3-methylhpet-1-ynyl,
   2-cyclopropylethynyl, 2-cyclobutylethynyl, 2-cyclopentylethynyl, 2-cyclohexylethynyl, 2-cycloheptylethynyl,
   ethynyl benzene, wherein the benzene ring is optionally substituted with an halogen.
4. The compound or a pharmaceutically acceptable salt or prodrug thereof according to statements 1 to 3, wherein R¹ is selected from the group consisting of:
   vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct-1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl, wherein said vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct-1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl is optionally substituted with OC₁₋₄alkyl, OH, halogen, or C₆₋₁₀aryl,
   ethynyl, prop-1-ynyl, buty-1-ynyl, pent-1-ynyl, 3-methylbut-1-ynyl, hex-1-ynyl, 4-methylpent-1-ynyl, 3-methylpent-1-ynyl, hept-1-ynyl, 5-methylhex-1-ynyl, 4-methylhex-1-ynyl, 3-methylhex-1-ynyl, 4,4-dimethylpent-1-ynyl, oct-1-ynyl, 6-methylhpet-1-ynyl, 5-methylhpet-1-ynyl, 4-methylhpet-1-ynyl, 3-methylhpet-1-ynyl,
   2-cyclopropylethynyl, 2-cyclobutylethynyl, 2-cyclopentylethynyl, 2-cyclohexylethynyl, 2-cycloheptylethynyl,
   ethynyl benzene, wherein the benzene ring is optionally substituted with an halogen.
5. The compound or a pharmaceutically acceptable salt or prodrug thereof according to statements 1 to 4, wherein R¹ is selected from the group consisting of:
   vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct-1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl, wherein said vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct-1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl is optionally substituted with methoxy, OH, F, Cl, Br, I, or phenyl,
   ethynyl, prop-1-ynyl, buty-1-ynyl, pent-1-ynyl, 3-methylbut-1-ynyl, hex-1-ynyl, 4-methylpent-1-ynyl, 3-methylpent-1-ynyl, hept-1-ynyl, 5-methylhex-1-ynyl, 4-methylhex-1-ynyl, 3-methylhex-1-ynyl, 4,4-dimethylpent-1-ynyl, oct-1-ynyl, 6-methylhpet-1-ynyl, 5-methylhpet-1-ynyl, 4-methylhpet-1-ynyl, 3-methylhpet-1-ynyl,
   2-cyclopropylethynyl, 2-cyclobutylethynyl, 2-cyclopentylethynyl, 2-cyclohexylethynyl, 2-cycloheptylethynyl,
   ethynyl benzene, wherein the benzene ring is optionally substituted with a F, Cl, Br or I.
6. The compound or a pharmaceutically acceptable salt or prodrug thereof according to statements 1 to 5, wherein R¹ is selected from the group consisting of: vinyl, vinylC₁₋₄alkyl, C₁₋₄alkylene-vinyl, ethynylC₁₋₄alkyl, ethynylC₃₋₅cycloalkyl, and ethynyl benzene.
7. The compound or a pharmaceutically acceptable salt or prodrug thereof according to statements 1 to 6, wherein R¹ is selected from the group consisting of:
   vinyl, vinylC₁₋₄alkyl or C₁₋₄alkylene-vinyl, wherein said vinyl, vinylC₁₋₄alkyl or C₁₋₄alkylene-vinyl are optionally substituted with a OC₁₋₄alkyl, OH, halogen, or C₆₋₁₀aryl, ethynylC₁₋₄alkyl, ethynylC₃₋₅cycloalkyl, and ethynyl benzene, wherein said ethynyl benzene ring is optionally substituted with a F, Cl, Br or I.
8. The compound or a pharmaceutically acceptable salt or prodrug thereof according to any one of statements 1 to 7, wherein R¹ is ethynyl.
9. The compound or a pharmaceutically acceptable salt or prodrug thereof according to any one of statements 1-8, wherein R¹ is ethynyl alkyl wherein the alkyl group is a straight or branched C1 to C3 chain.
10. The compound or a pharmaceutically acceptable salt or prodrug thereof according to any one of statements 1 to 9, wherein R¹ ethynyl cycloalkyl, wherein the cycloalkyl is a C3, C4 or C5 cycloalkyl.
11. The compound or a pharmaceutically acceptable salt or prodrug thereof according to any one of statements 1 to 10, wherein R¹ is ethynyl benzene substituted with an halogen.
12. The compound or a pharmaceutically acceptable salt or prodrug thereof according to any one of statements 1 to 11, wherein R² is selected from the group consisting of: an optionally substituted C2-C4 chain comprising a vinyl or ethynyl group, F, Cl, Br and I.
13. The compound or a pharmaceutically acceptable salt or prodrug thereof according to any one of statements 1 to 12, wherein R² is selected from the group consisting of: vinyl, prop-1-enyl, allyl, 3-methylbut-1-enyl, but-1-enyl, but-2-enyl, but-3-enyl, pent-1-enyl, pent-2-enyl, pent-3-enyl, pent-4-enyl, hex-1-enyl, hex-2-enyl, hex-3-enyl, hex-4-enyl, hex-5-enyl, 4-methylpent-1-enyl, 4-methylpent-2-enyl, ethynyl, prop-1-ynyl, prop-2-ynyl, 3-methylbut-1-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 4-methylpent-1-ynyl, 4-methylpent-2-ynyl, wherein said vinyl, prop-1-enyl, allyl, 3-methylbut-1-enyl, but-1-enyl, but-2-enyl, but-3-enyl, pent-1-enyl, pent-2-enyl, pent-3-enyl, pent-4-enyl, hex-1-enyl, hex-2-enyl, hex-3-enyl, hex-4-enyl, hex-5-enyl, 4-methyl pent-1-enyl, 4-methylpent-2-enyl, ethynyl, prop-1-ynyl, prop-2-ynyl, 3-methylbut-1-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 4-methylpent-1-ynyl, 4-methylpent-2-ynyl, are optionally substituted with a halogen, OH or CH₃, and
   a halogen.
14. The compound or a pharmaceutically acceptable salt or prodrug thereof according to any one of statements 1 to 13, wherein R² is selected from the group consisting of: vinyl, vinylC₁₋₄alkyl, C1-alkylene-vinyl, ethynyl, ethynylC₁₋₄alkyl, C₁₋₄alkylene-ethynyl, F, Cl, Br and I; wherein said vinyl, vinylC₁₋₄alkyl, C₁₋₄alkylene-vinyl, ethynyl, ethynylC₁₋₄alkyl, C₁₋₄alkylene-ethynyl, are optionally substituted with a C₃₋₆cycloalkyl or a benzene ring, wherein the benzene ring is optionally substituted with a halogen, OH or CH₃.
15. The compound or a pharmaceutically acceptable salt or prodrug thereof according to any one of statements 1 to 14 wherein R¹ is selected from the group consisting of:

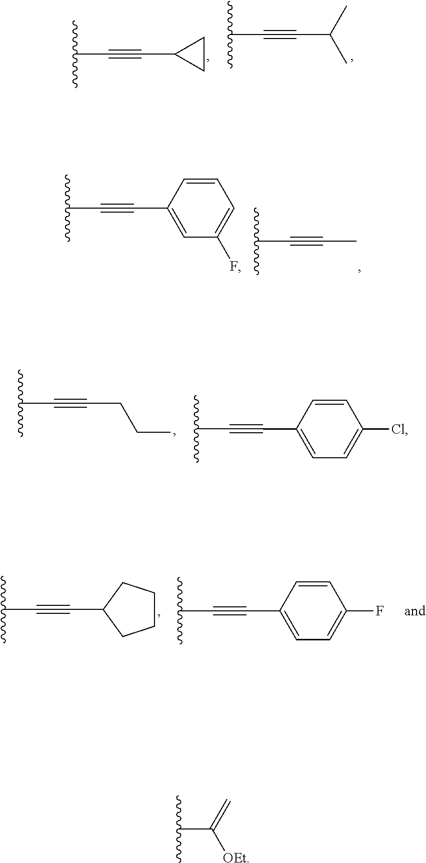

16. The compound or a pharmaceutically acceptable salt or prodrug thereof according to any one of statements 1 to 15, wherein R² is fluor.
17. The compound or a pharmaceutically acceptable salt or prodrug thereof according to any one of statements 1 to 16, wherein R² is a C2-C4 chain comprising a vinyl or ethynyl group, optionally substituted with a C3-C6 cycloalkyl.
18. The compound or a pharmaceutically acceptable salt or prodrug thereof, according to any one of statement 1 to 17, wherein R² is a vinyl or ethynyl substituted with a C3-C6 cycloalkyl.
19. The compound or a pharmaceutically acceptable salt or prodrug thereof according to any one of statements 1 to 18, wherein R² is a vinyl or ethynyl.
20. The compound or a pharmaceutically acceptable salt or prodrug thereof, according to statement 19, wherein the cycloalkyl is cyclopropyl.
21. The compound or a pharmaceutically acceptable salt or prodrug thereof according any one of statements 1 to 20, wherein R² is selected from ethynyl, vinyl and cyclopropylethynyl.
22. A pharmaceutical composition comprising:
   a compound according to any one of statements 1 to 21, or a pharmaceutically acceptable salt, and
   at least one pharmaceutical acceptable carrier.
23. A compound according to any one of statements 1 to 21 or a pharmaceutical composition according to statement 22, for use as a medicament.
24. A compound according to any one of statements 1 to 21 or a pharmaceutical composition according to statement 22, for use in the treatment or prevention of vial infections.
25. The compound for use or pharmaceutical composition for use according to statement 24, wherein the viral infection is an infection by an RNA virus.
26. The compound for use or pharmaceutical composition for use according to statement 25, wherein the RNA virus is selected from the group consisting of coronavirus, measles, tacaribe virus, yellow fever virus, influenzavirus, Chikungunya, dengue, respiratory syncytial virus (RSV), human immunodeficiency virus (HIV) and norovirus.
27. The compound for use or pharmaceutical composition for use according to any one of statements 25 or 27, wherein the RNA virus is selected from the group consisting of coronavirus, measles, tacaribe virus, yellow fever virus, influenzavirus A, influenzavirus B, Chikungunya, dengue, respiratory syncytial virus (RSV), human immunodeficiency virus (HIV) and norovirus.
28. The compound for use or a pharmaceutical composition for use according to any one of statements 25 to 28, for use in the treatment or prevention of an RNA virus, wherein the RNA virus is selected from the group consisting of a coronavirus, measles, tacaribe virus, yellow fever virus, influenza virus, and norovirus.
29. The compound for use or a pharmaceutical composition for use according to any one of statements 26 to 28, wherein the coronavirus is MERS or Sars-Cov2.
30. A method of treating and/or preventing a viral infection, comprising the step of administering to an individual a compound with general structure (I) or (II) or a pharmaceutically acceptable salt or prodrug thereof according to any one of statements 1 to 21.
31. A compound with general structure (I) or a pharmaceutically acceptable salt thereof

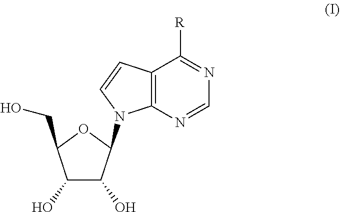

for use in the treatment or prevention of an RNA virus,
wherein R is selected from the group consisting of:
   ethynyl or an ethynyl alkyl, wherein the alkyl group is a straight or branched C1 to C6 chain,
   ethynyl cycloalkyl, wherein the cycloalkyl is a C3-C7 cycloalkyl,
and
   ethynyl benzene, wherein the benzene ring is optionally substituted with an halogen (e.g. F or Cl), OH or CH₃, or wherein the benzene ring is optionally substituted with an halogen (e.g. F or Cl)
32. The compound or a pharmaceutically acceptable salt thereof according to statement 31, for use in the treatment or prevention of an RNA virus, wherein R is ethynyl.
33. The compound or a pharmaceutically acceptable salt thereof according to statement 31, for use in the treatment or prevention of an RNA virus, wherein R is ethynyl alkyl wherein the alkyl group is a straight or branched C1 to C3 chain.
34. The compound or a pharmaceutically acceptable salt thereof according to statement 31, for use in the treatment or prevention of an RNA virus, wherein R ethynyl cycloalkyl, wherein the cycloalkyl is a C3, C4 or C5 cycloalkyl.
35. The compound or a pharmaceutically acceptable salt thereof according to statement 31, for use in the treatment or prevention of an RNA virus, wherein R is ethynyl benzene substituted with an halogen.
36. The compound or a pharmaceutically acceptable salt thereof according to statement 31, for use in the treatment or prevention of an RNA virus, wherein the RNA virus is selected from the group consisting of a coronavirus, measles, tacaribe virus, yellow fever virus, influenza virus, and norovirus.
37. The compound or a pharmaceutically acceptable salt thereof according to

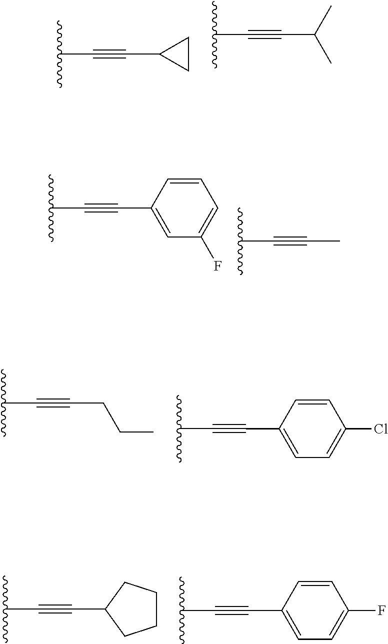

statement 31, for use in the treatment or prevention of an RNA virus, wherein the RNA virus is selected from the group consisting of a coronavirus, measles, tacaribe virus, yellow fever virus, influenza virus, and norovirus, and wherein R is selected from the group consisting of:
38. A compound with general structure (I) or a pharmaceutically acceptable salt thereof

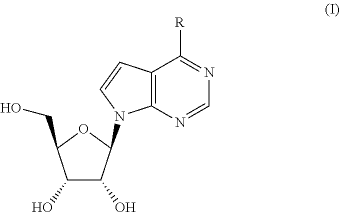

wherein R is selected from the group consisting of:
ethynyl or an ethynyl alkyl, wherein the alkyl group is a straight or branched C1 5 to C6 chain,
ethynyl cycloalkyl, wherein the cycloalkyl is a C3-C7 cycloalkyl,
and
ethynyl benzene, wherein the benzene ring is optionally substituted with an halogen (e.g. F or Cl), OH or CH₃, or wherein the benzene ring is optionally substituted with an halogen (e.g. F or Cl).
39. The compound or a pharmaceutically acceptable salt thereof according to statement 38, wherein R is ethynyl.
40. The compound or a pharmaceutically acceptable salt thereof according to statement 38, wherein R is ethynyl alkyl wherein the alkyl group is a straight or branched C1 to C3 chain.
41. The compound or a pharmaceutically acceptable salt thereof according to statement 38, wherein R ethynyl cycloalkyl, wherein the cycloalkyl is a C3, C4 or C5 cycloalkyl.
42. The compound or a pharmaceutically acceptable salt thereof according to statement 38, wherein R is ethynyl benzene substituted with an halogen.
43. The compound or a pharmaceutically acceptable salt thereof according to statement 38, wherein R is selected from the group consisting of:

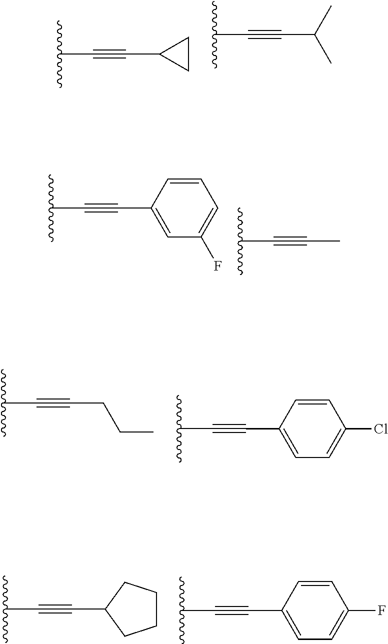

44. The compound according to any one of statements 38 to 43, for use as a medicament.
45. A pharmaceutical composition comprising:
   a compound according to any one of statements 38 to 43, or a pharmaceutically acceptable salt, and
   a pharmaceutical acceptable carrier.
46. A method of treating and/or preventing a viral infection, comprising the step of administering to an individual a compound according to any one of statements 38-43, or a pharmaceutically acceptable salt
47. A compound with general formula (I), or a pharmaceutically acceptable salt thereof

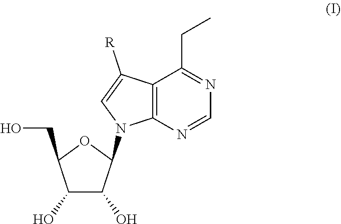

wherein R is:
a C2-C6 chain comprising a vinyl or ethynyl group, the C2-C6 chain optionally substituted with a C3-C6 cycloalkyl, or the C2-C6 chain optionally substituted with a benzene ring wherein the benzene ring is optionally substituted with a halogen, OH or CH3, or
a halogen
48. The compound according to statement 47, or a pharmaceutically acceptable salt thereof, wherein R is fluor.
49. The compound according to statement 47, or a pharmaceutically acceptable salt thereof wherein R is a C2-C4 chain comprising a vinyl or ethynyl group, optionally substituted with a C3-C6 cycloalkyl.
50. The compound according to statement 47 or 49, or a pharmaceutically acceptable salt thereof, wherein R is a vinyl or ethynyl substituted with a C3-C6 cycloalkyl.
51. The compound according to statement 47 or 49, or a pharmaceutically acceptable salt thereof, wherein R is a vinyl or ethynyl.
52. The compound according to statement 47, 49 or 50, or a pharmaceutically acceptable salt thereof, wherein the cycloalkyl is cyclopropyl.
53. The compound according to statement 47 or any one of statements 47 to 52, or a pharmaceutically acceptable salt thereof, wherein R is selected from ethynyl, vinyl and cyclopropylethynyl.
54. A compound according to any one of statements 49 to 53, or a pharmaceutically acceptable salt thereof, for use as a medicament.
55. A compound according to any one of statements 49 to 53, or a pharmaceutically acceptable salt thereof, for use in the treatment or prevention of a viral infection.
56. The compound or a pharmaceutically acceptable salt thereof, for use in accordance to statement 55, wherein the viral infection is a RNA virus viral infection.
57. The compound or a pharmaceutically acceptable salt thereof, for use in accordance to statement 56, wherein the RNA virus is selected from the group consisting of norovirus, influenza A, influenza B, coronavirus, Chikungunya, dengue, yellow fever, measles, tacaribe, RSV and HIV.
58. The compound or a pharmaceutically acceptable salt thereof, for use in accordance to statement 57, wherein the coronavirus is MERS or Sars-Cov2.
59. The compound or a pharmaceutically acceptable salt thereof, for use in accordance to statement 58, wherein R is fluor and the RNA virus is influenza, chikungunya, dengue or MERS.
60. A pharmaceutical comprising a compound according to any one of statements 47 to 53, or a pharmaceutically acceptable salt thereof, and a pharmaceutically acceptable carrier.
61. A method of treating or preventing a viral infection comprising the step of administering to an individual an affective amount of a compound according to any one of statements 47 to 53, or a pharmaceutically acceptable salt thereof.

### DETAILED DESCRIPTION

FIG. 1. Structure of tubercidin and some 7-deazaadenosine derivatives with potential antiviral activity.

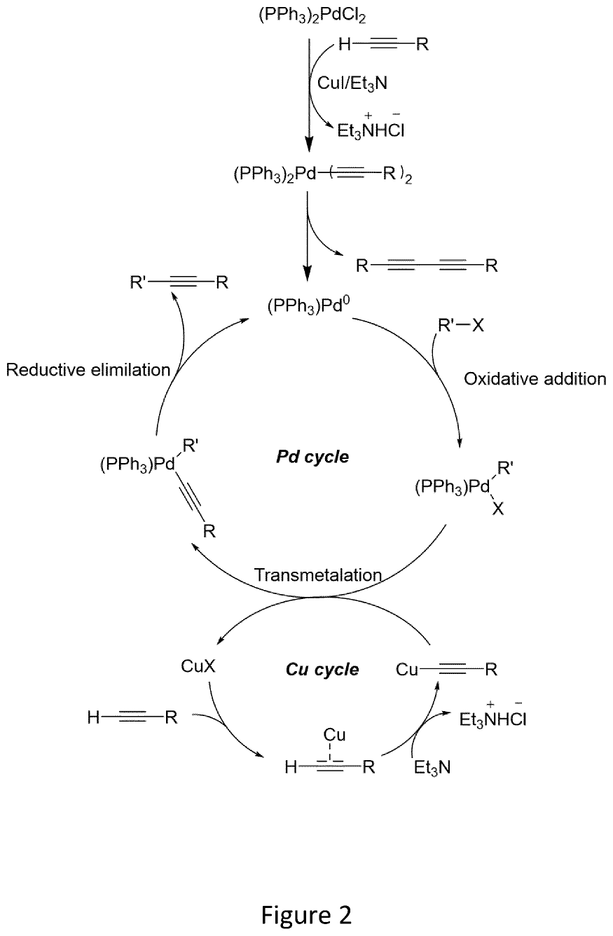

FIG. 2. mechanism for the copper co-catalysed Sonogashira reaction.17,18

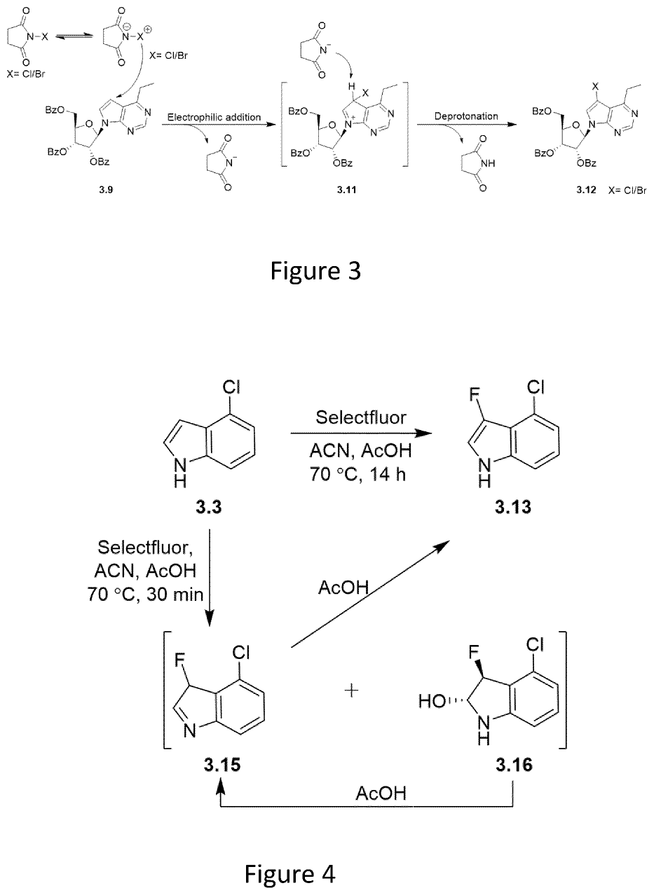

FIG. 3. mechanism of the electrophilic halogenation with NCS/NBS.

FIG. 4. Mechanism of the electrophilic fluorination.¹⁹

Before the present invention is described, it is to be understood that this invention is not limited to particular processes, methods, and compounds described, as such processes, methods, and compounds may, of course, vary. It is also to be understood that the terminology used herein is not intended to be limiting, since the scope of the present invention will be limited only by the appended claims.

When describing the compounds and processes of the invention, the terms used are to be construed in accordance with the following definitions, unless a context dictates otherwise.

As used in the specification and the appended claims, the singular forms “a”, “an,” and “the” include both singular and plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

When describing the present invention, the terms used are to be construed in accordance with the following definitions, unless a context dictates otherwise.

The terms described above and others used in the specification are well understood to those in the art.

The term “pharmaceutically acceptable carrier or excipient” as used herein in relation to pharmaceutical compositions and combined preparations means any material or substance with which the active principle i.e. the compounds of general formula (A), and optionally an antiviral agent and/or an immunosuppressant or immunomodulator may be formulated in order to facilitate its application or dissemination to the locus to be treated, for instance by dissolving, dispersing or diffusing said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Suitable pharmaceutical carriers for use in said pharmaceutical compositions and their formulation are well known to those skilled in the art. There is no particular restriction to their selection within the present invention. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation from a reaction mixture. Where groups can be substituted, such groups may be substituted with one or more, and preferably one, two or three substituents.

The terminology “optionally substituted” as used herein is meant to indicate that a group may be unsubstituted or substituted.

“Alkyl” means a straight-chain or branched hydrocarbon chain with up to 6 carbon atoms. Each hydrogen of an alkyl carbon may be replaced by a substituent as further specified herein.

“Alkenyl” means a straight-chain or branched hydrocarbon chain that contains at least one carbon-carbon double bond. Each hydrogen of an alkenyl carbon may be replaced by a substituent as further specified herein.

“Alkynyl” means a straight-chain or branched hydrocarbon chain that contains at least one carbon-carbon triple bond. Each hydrogen of an alkynyl carbon may be replaced by a substituent as further specified herein.

“C1-3 alkyl” means an alkyl chain having 1-3 carbon atoms, e.g. if present at the end of a molecule: methyl, ethyl, n-propyl, isopropyl, or e.g. —CH2-, —CH2-CH2-, —CH(CH3)-, —CH2-CH2-CH2-, —CH(C2H5)-, —C(CH3)2-, when two moieties of a molecule are linked by the alkyl group. Each hydrogen of a C1-3 alkyl carbon may be replaced by a substituent as further specified herein.

“C1-4 alkyl” means an alkyl chain having 1-4 carbon atoms, e.g. if present at the end of a molecule: methyl, ethyl, n-propyl, isopropyl, n-butyl, isobutyl, sec-butyl, tert-butyl, or e.g. —CH2-, —CH2-CH2-, —CH(CH3)-, —CH2-CH2-CH2-, —CH(C2H5)-, —C(CH3)2-, when two moieties of a molecule are linked by the alkyl group. Each hydrogen of a C1-4 alkyl carbon may be replaced by a substituent as further specified herein.

“C1-6 alkyl” means an alkyl chain having 1-6 carbon atoms, e.g. if present at the end of a molecule: C1-4 alkyl, methyl, ethyl, n-propyl, isopropyl, n-butyl, isobutyl, sec-butyl; tert-butyl, n-pentyl, n-hexyl, or e.g. —CH2-, —CH2-CH2-, —CH(CH3)-, —CH2-CH2-CH2-, —CH(C2H5)-, —C(CH3)2-, when two moieties of a molecule are linked by the alkyl group. Each hydrogen of a C1-6 alkyl carbon may be replaced by a substituent as further specified herein. The term “C1-12 alkyl” is defined accordingly.

When the suffix “ene” is used in conjunction with an alkyl group, i.e. “alkylene”, this is intended to mean the alkyl group as defined herein having two single bonds as points of attachment to other groups. As used herein, the term “C1-6alkylene”, by itself or as part of another substituent, refers to C1-6alkyl groups that are divalent, i.e., with two single bonds for attachment to two other groups. Alkylene groups may be linear or branched and may be substituted as indicated herein. Non-limiting examples of alkylene groups include methylene (—CH2-), ethylene (—CH2-CH2-), methylmethylene (—CH(CH3)-), 1-methyl-ethylene (—CH(CH3)-CH2-), n-propylene (—CH2-CH2-CH2-), 2-methylpropylene (—CH2-CH(C13)-CH2-), 3-methylpropylene (—CH2-CH2-CH(CH3)-), n-butylene (—CH2-CH2-CH2-CH2-), 2-methylbutylene (—CH2-CH(CH3)-CH2-CH2-), 4-methylbutylene (—CH2-CH2-CH2-CH(CH3)-), pentylene and its chain isomers, hexylene and its chain isomers.

“C2-6 alkenyl” means an alkenyl chain having 2 to 6 carbon atoms, e.g. if present at the end of a molecule: —CH═CH2, —CH═CH—CH3, —CH2-CH═CH2, —CH═CH—CH2-CH3, —CH═CH—CH═CH2, or e.g. —CH═CH—, when two moieties of a molecule are linked by the alkenyl group. Each hydrogen of a C2-6 alkenyl carbon may be replaced by a substituent as further specified herein. The term “C2-12 alkenyl” is defined accordingly.

“C2-6 alkynyl” means an alkynyl chain having 2 to 6 carbon atoms, e.g. if present at the end of a molecule: —C═CH, —CH2-C═CH, CH2-CH2-C═CH, CH2-C═C—CH3, or e.g. —C═C— when two moieties of a molecule are linked by the alkynyl group. Each hydrogen of a C2-6 alkynyl carbon may be replaced by a substituent as further specified herein. The term “C2-12 alkynyl” is defined accordingly.

As used herein with respect to a substituting radical, and unless otherwise stated, the term “acyl” broadly refers to a substituent derived from an acid such as an organic monocarboxylic acid, a carbonic acid, a carbamic acid (resulting into a carbamoyl substituent) or the thioacid or imidic acid (resulting into a carbamidoyl substituent) corresponding to said acids, and the term “sulfonyl” refers to a substituent derived from an organic sulfonic acid, wherein said acids comprise an aliphatic, aromatic or heterocyclic group in the molecule.

As used herein with respect to a substituting radical, and unless otherwise stated, the term “cycloalkyl” means a mono- or polycyclic saturated hydrocarbon monovalent radical, such as for instance cyclopropyl, cyclobutyl, cyclopentyl, cyclohexyl, cycloheptyl, cyclooctyl and the like, or a C7-10 polycyclic saturated hydrocarbon monovalent radical having from 7 to 10 carbon atoms such as, for instance, norbornyl, fenchyl, trimethyltricycloheptyl or adamantyl. By way of example, “C3-7 cycloalkyl” or “C3-7 cycloalkyl ring” means a cyclic alkyl chain having 3-7 carbon atoms, e.g. cyclopropyl, cyclobutyl, cyclopentyl, cyclohexyl, cyclohexenyl, cycloheptyl. Preferably, cycloalkyl refers to cyclopropyl, cyclobutyl, cyclopentyl, cyclohexyl, or cycloheptyl. Each hydrogen of a cycloalkyl carbon may be replaced by a substituent as specified herein.

As used herein with respect to a substituting radical, and unless otherwise stated, the term “aryl” designate any mono- or polycyclic aromatic monovalent hydrocarbon radical having from 6 up to 30 carbon atoms such as but not limited to phenyl, naphthyl, anthracenyl, phenantracyl, fluoranthenyl, chrysenyl, pyrenyl, biphenylyl, terphenyl, picenyl, indenyl, biphenyl, indacenyl, benzocyclobutenyl, benzocyclooctenyl and the like, including fused benzo-C4-β cycloalkyl radicals (the latter being as defined above) such as, for instance, indanyl, tetrahydronaphthyl, fluorenyl and the like, all of the said radicals being optionally substituted with one or more substituents independently selected from the group consisting of halogen, amino, trifluoromethyl, hydroxyl, sulfhydryl and nitro, such as for instance 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein, e.g. with respect to a substituting radical such as the combination of substituents in certain positions of the compounds, and unless otherwise stated, the term “homocyclic” means a mono- or polycyclic, saturated or mono unsaturated or polyunsaturated hydrocarbon radical having from 4 up to 15 carbon atoms but including no heteroatom in the said ring; for instance said combination of substituents may form a C2-6 alkylene radical, such as tetramethylene, which cyclizes with the carbon atoms in certain positions of the thiazolo[5,4-d]pyrimidine, oxazolo[5,4-d]pyrimidine, thieno[2,3-d]pyrimidine or purine ring.

As used herein, the term “vinyl” refers to the ethenyl group, —CH═CH₂.

As used herein, the term “vinylalkyl” means a vinyl group as defined herein wherein one hydrogen atom is replaced with an alkyl as defined herein.

As used herein, the term “alkylene-vinyl” means an alkylene group as defined herein wherein one hydrogen atom is replaced with a vinyl as defined herein.

As used herein, the term “ethynyl” refers to the group, —CH═CH₂.

As used herein, the term “ethynyl alkyl” means an ethynyl group as defined herein wherein one hydrogen atom is replaced with an alkyl as defined herein.

As used herein, the term “alkylene ethynyl” means an alkylene group as defined herein wherein one hydrogen atom is replaced with an ethynyl group as defined herein.

As used herein, the term “ethynyl cycloalkyl” means an ethynyl group as defined herein wherein one hydrogen atom is replaced with an cycloalkyl as defined herein.

As used herein, the term “ethynyl benzene” means an ethynyl group as defined herein wherein one hydrogen atom is replaced with benzene ring.

As used herein, the term “a C2-C6 chain comprising a vinyl or ethynyl group” refers to a straight or branched hydrocarbon chain, wherein the chain comprises a double bond (vinyl group) or a triple bond (ethynyl group).

The term “halo” or “halogen” as a group or part of a group is generic for fluoro, chloro, bromo, iodo.

The term “amino” refers to the group —NH₂.

The term “dimethylamino” refers to the group —N(CH₃)₂.

Where a compound of general structure (I) or (II) contains an vinyl or ethynyl group, geometric cis/trans (or Z/E) isomers are possible.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

As used herein and unless otherwise stated, the term “solvate” includes any combination which may be formed by derivative of this invention with a suitable inorganic solvent (e.g. hydrates) or organic solvent, such as but not limited to alcohols, ketones, esters, ethers, nitriles and the like.

The term “individual” as used herein refers to a mammal. The individual will preferably be a human, but may also be a domestic livestock, laboratory or pet animals.

Pharmaceutically acceptable salts of the compounds of general structure (I) or (II) include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2 napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

Pharmaceutically acceptable salts of compounds of general structure (I) or (II) may be prepared by one or more of these methods:
(i) by reacting the compound of general structure (I) or (II) with the desired acid;
(ii) by reacting the compound of general structure (I) or (II) with the desired base;
(iii) by removing an acid- or base-labile protecting group from a suitable precursor of the compound of general structure (I) or (II) or by ring-opening a suitable cyclic precursor, for example, a lactone or lactam, using the desired acid; or
(iv) by converting one salt of the compound of general structure (I) or (II) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quatemized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

In some embodiments, the present invention relates to the use of at least one compound of general structure (I) or (II), in (the preparation of a composition for) the treatment and/or prevention of viral infections.

The invention also generally covers all pharmaceutically acceptable prodrugs or “pre-drugs” of the compounds of formula (I) or (II) for which general reference is made to the prior art cited hereinbelow.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

In some embodiments, the prodrugs of the compounds of general structure (I) or (II) are ester derivatives.

In some embodiments, the present invention relates to a method of treatment and/or prevention of viral infections, comprising administering to a subject in need thereof an effective amount of at least one compound of general structure (I) or (II), or a pharmaceutical composition comprising said at least one compound of general structure (I) or (II).

In some embodiments, the present invention relates to the use of at least one compound of general structure (I) or (II), in (the preparation of a composition for) the treatment and/or prevention of viral infections, preferably viral infections caused by an RNA virus, more preferably viral infections caused by coronavirus, measles, tacaribe virus, yellow fever virus, influenzavirus, Chikungunya, dengue, respiratory syncytial virus (RSV), human immunodeficiency virus (HIV) and norovirus.

The invention further provides pharmaceutical compositions that include effective amounts of compounds of general structure (I) or (II), or pharmaceutically accepted salts thereof, and at least one pharmaceutically acceptable carrier. The compounds of general structure (I) or (II) or pharmaceutically acceptable salts thereof, are as herein described.

The compounds according to the invention may be administered as the sole active ingredient or together, i.e. in a fixed or free combination, with other therapeutic agents used in clinical practice for the treatment of those diseases listed above.

The compounds according to the invention and the other pharmaceutical active agent(s) may be administered together or separately and, when administered separately, administration may occur simultaneously or sequentially, in any order. The amounts of the compounds according to the invention and the other pharmaceutically active agent (s) and the relative timings of administration will be selected in order to achieve the desired combined therapeutic effect. The administration in combination of a compound of formula (I) or a stereoisomer, tautomer, racemic, salt, hydrate or solvate thereof, with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. Nos. 6,372,778, 6,369,086, 6,369,087 and 6,372,733.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. Nos. 6,372,778, 6,369,086, 6,369,087 and 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcelIulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, disintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical formulation according to the invention, it can be advantageous to employ α-, β- or γ-cyclodextrins or their derivatives. In addition, co-solvents such as alcohols may improve the solubility and/or the stability of the compounds. In the preparation of aqueous compositions, addition of salts of the compounds of the invention can be more suitable due to their increased water solubility.

Appropriate cyclodextrins are α-, β- or γ-cyclodextrins (CDs) or ethers and mixed ethers thereof wherein one or more of the hydroxyl groups of the anhydroglucose units of the cyclodextrin are substituted with alkyl, particularly methyl, ethyl or isopropyl, e.g. randomly methylated β-CD; hydroxyalkyl, particularly hydroxyethyl, hydroxypropyl or hydroxybutyl; carboxyalkyl, particularly carboxymethyl or carboxyethyl; alkylcarbonyl, particularly acetyl; alkoxycarbonylalkyl or carboxyalkoxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD). The term mixed ether denotes cyclodextrin derivatives wherein at least two cyclodextrin hydroxyl groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors. In particular, the present invention encompasses a pharmaceutical formulation comprising an effective amount of a compound according to the invention with a pharmaceutically acceptable cyclodextrin. The present invention also encompasses cyclodextrin complexes consisting of a compound according to the invention and a cyclodextrin.

Particular reference is made to the compositions, formulations (and carriers, excipients, diluents, etc. for use therein), routes of administration etc., which are known per se such as those described in U.S. Pat. No. 4,997,834 and EP-A-0 370 498.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products, and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded, or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides, and derivatives thereof.

The preparations may be prepared in a manner known per se, which usually involves mixing the at least one compound according to the invention with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

The pharmaceutical formulations of the invention are preferably in a unit dosage form, and may be suitably packaged, for example in a box, blister, vial, bottle, sachet, ampoule or in any other suitable single-dose or multi-dose holder or container (which may be properly labelled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the formula (I) above that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

In accordance with the method of the present invention, said pharmaceutical formulation can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. For subcutaneous or intravenous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

The compositions are of value in the veterinary field, which for the purposes herein not only includes the prevention and/or treatment of diseases in animals, but also
for economically important animals such as cattle, pigs, sheep, chicken, fish, etc.
enhancing the growth and/or weight of the animal and/or the amount and/or the quality of the meat or other products obtained from the animal. Thus, in a further aspect, the invention relates to a (pharmaceutical) formulation for veterinary use that contains at least one compound of the invention and at least one suitable carrier (i.e. a carrier suitable for veterinary use). The invention also relates to the use of a compound of the invention in the preparation of such a formulation.

The following examples are provided for the purpose of illustrating the present invention and by no means should be interpreted to limit the scope of the present invention.

### Examples

### Example 1. General Synthesis of 6-substituted 7-deazapurine Ribonudeoside Analogues

The 7-deazapurine ribonucleoside scaffold was obtained from 7-iodo-6-chloro-7-deazapurine and fully protected D-ribose via a Vorbrüggen glycosylation reaction according to a literature procedure (Schemel).^{14,15} Upon treatment with N-iodosuccinimide (NIS), compound 3.3 was converted to 7-iodo-6-chloro-7-deazapurine (3.4) via halogenation. Benzoylated D-ribose 3.5 was synthesized from D-ribose over three steps in good yield (59%).¹⁶ After a Vorbrüggen glycosylation reaction, the 7-iodo-6-chloro-7-deazapurine ribonucleoside analogue 3.6 was obtained. In the one-pot glycosylation reaction, the use of 7-iodine nucleobase 3.4 showed good stereoselectivity and yield. This might be due to the function of the 7-iodine substituent as an electron-withdrawing group to increase the reactivity of the pyrrole nitrogen. Then, the iodine atom at the 7-position was removed by treatment with iPrMgCl·LiCl via halogen-magnesium exchange to give the key intermediate 3.7 which was used to synthesize a series of 6-substituted 7-deazapurine ribonucleoside and a series of 7-substituted 6-ethyl-7-deazapurine ribonucleoside analogues.

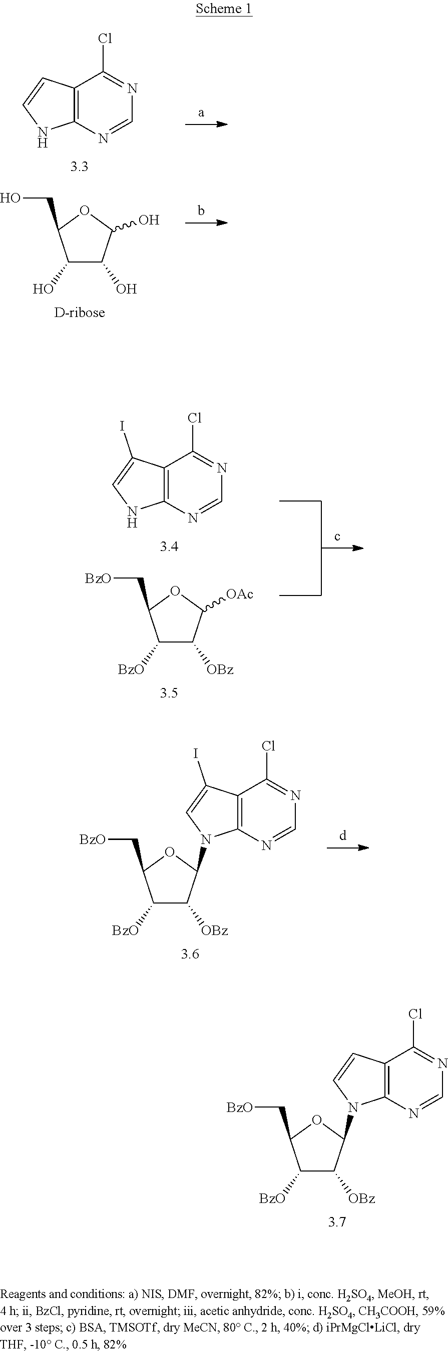

As shown in Scheme 2, the key intermediate 3.7 was converted to the corresponding 6-cyclopropyl analogue by treatment with a cyclopropyl Grignard reagent via a Fe/Cu-catalysed coupling reaction, followed by removing all benzoyl protecting groups to yield reference compound 3.1a in 65% over two steps.⁵ The synthesis of the other 6-substituted 7-deazapurine ribonucleoside analogues was mainly accomplished via palladium-catalysed coupling reactions, including Stille and Sonogashira reactions. A 1-ethoxyvinyl group was introduced at the 6-position of compound 3.7 via Stille reaction using Pd(PPh₃)₂Cl₂ and tributyl(1-ethoxyvinyl)tin and all benzoyl protecting groups were removed to obtain compound 3.1b. Moreover, a range of alkyl- and arylacetylenes was coupled with fully protected 6-chloro-7-deazapurine ribonucleoside via Sonogashira reaction, followed by removed its benzoyl groups to give compounds 3.1c-j in good yields (51%-75% over 2 steps). As shown in FIG. 2, The proposed mechanism is composed of a Pd(0)-catalysed cycle and a Cu-catalysed cycle.^{17,18} After activation of the pre-catalyst, aryl halide was reacted with the Pd(0) catalyst via a oxidative addition to produce a Pd(II) intermediate. Then, the copper acetylide provided by the Cu-catalysed cycle is used to transfer alkyl- and arylethynyl groups to the palladium(II) centre during the transmetalation and regenerate the Cu catalyst. Finally, the reductive elimination leads to the coupling product and the active Pd(0)

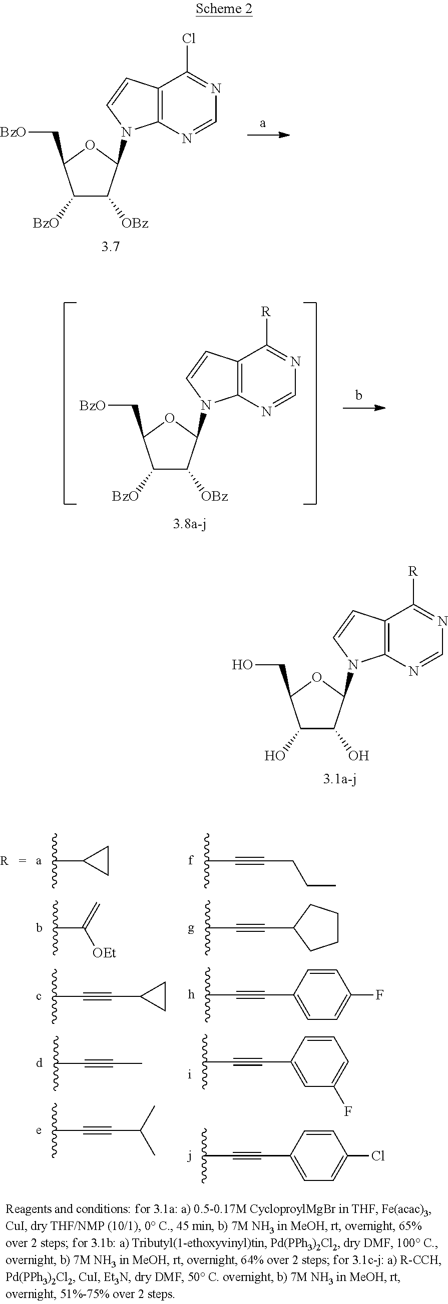

### Example 2. Synthesis of 7-substituted 6-ethyl-7-deazapurine Ribonudeoside Analogues

6-Ethyl-7-deazapurine ribonucleoside derivative 3.9 was prepared from key intermediate 3.7 upon treatment with ethylmagnesium bromide, Fe(acac)₃ and CuI. Subsequently, compound 3.9 underwent a substitution reaction by treatment with NIS to afford iodide 3.10, which was then used in palladium-catalysed coupling reactions such as Stille and Sonogashira reactions to synthesize 7-substituted 6-ethyl-7-deazapurine ribonucleoside analogues (Scheme 3, 3.11-n). Via a Sonogashira reaction catalysed by Pd(PPh₃)₂Cl₂ and a deprotection reaction⁸, compound 3.10 was converted to 7-ethynyl analogue 3.11 over three steps in good yield (67%). A cyclopropylacetylene reagent was used for the preparation of compound 3.1n. The vinyl group was introduced at the 7-position via Stille reaction and all benzoyl groups were removed with 7M NH₃ in MeOH to obtain compound 3.1m.

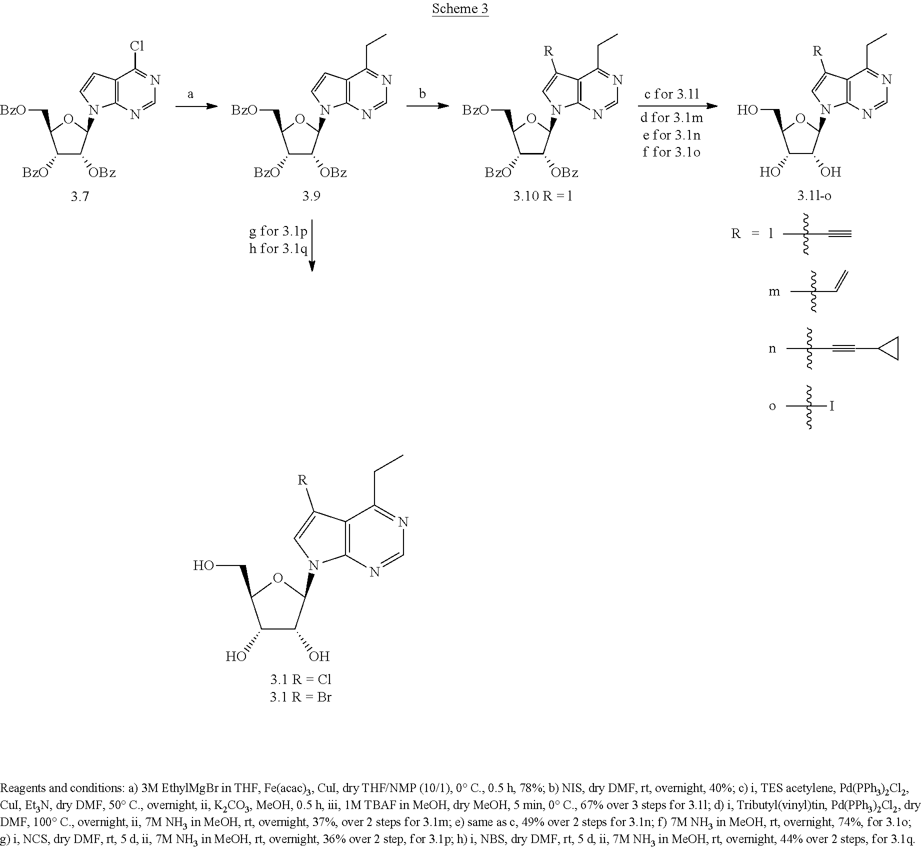

7-Halo-6-ethyl-7-deazapurine ribonucleoside analogues (3.10-r, Scheme 3 and Scheme 4) were synthesized from intermediate 3.9. After removal of all benzoyl groups of intermediate 3.10, the 7-iodo analogue 3.10 was obtained. N-chlorosuccinimide (NCS) and N-bromosuccinimide (NBS) were used respectively to introduce chlorine and bromine atoms at the 7-position of compound 3.9 via a halogenation. Via an electrophilic addition, compound 3.9 was converted to intermediate 3.11, followed by deprotonation to afford 7-halo derivatives (3.12, FIG. 3). After deprotection of all benzoyl groups, 7-chloro and 7-bromo analogues (3.1p and 3.1q) were obtained. The 7-fluoro-6-ethyl analogue 3.1r was synthesized from 7-fluoro-6-chloro-7-deazapurine ribonucleoside derivative 3.14 (Scheme 4) which was prepared according to a literature procedure¹⁹. Selectfluor as a fluorine donor was used to introduce a fluorine atom at the 5-position of 3.3 by an electrophilic fluorination to give intermediates 3.15 and 3.16 which performed dehydration in the present of acetic acid at 70° C. to afford 4-chloro-5-fluoropyrrolo[2,3-d]pyrimidine 3.13 (FIG. 4). Compound 3.14 was obtained from 3.13 in a one-pot glycosylation reaction. Via a Fe/Cu-catalysed coupling reaction, a chlorine at the 6-position was replaced with an ethyl group and all benzoyl groups were removed to give compound 3.1r.

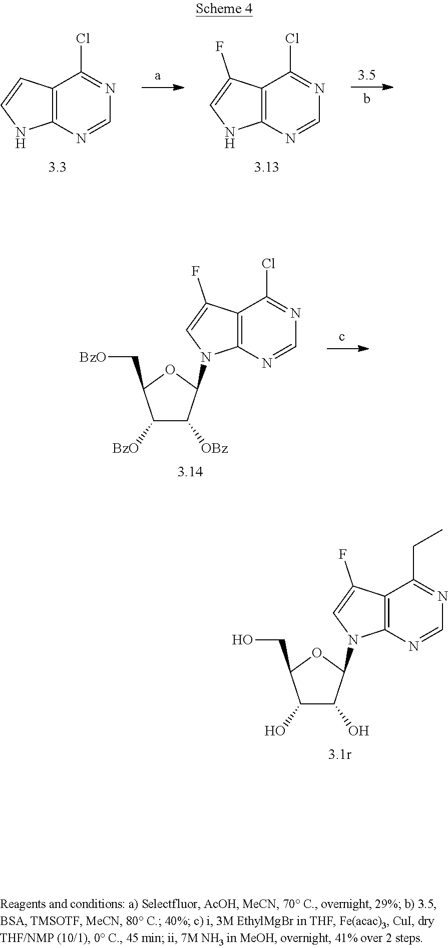

### Example 3. Anti-RNA Virus Activity

All synthesized 6-substituted and 7-substituted 6-ethyl ribonucleoside 7-deazaadenosine analogues were evaluated for antiviral activity (express as EC₅₀) and corresponding cytotoxicity (CC₅₀) against five representative RNA viruses, including MERS-CoV (Middle East respiratory syndrome coronavirus), measles virus, influenza A (H1N1) virus, tacaribe virus and yellow fever virus. Moreover, 6-substituted derivatives were tested against human norovirus.

### 3.1. Antiviral Activity Against Various RNA Viruses

Antiviral activity and cytotoxicity of reference compound 3.1a and compounds of the invention 3.1b-j and 3.1l-r were investigated against MERS-CoV (EMC strain, in Vero 76 cell line), measles virus (CC strain, in Vero 76 cell line), tacaribe virus (TRVL11573 strain, in Vero cell line) and yellow fever virus (YFV 17D strain, in Huh7 cell line), as well as M128533, 2′-fluoro-2′-deoxycytidine, ribavirin and infergen as positive controls, respectively. Screening against MERS-CoV showed that compounds 3.1b, 3.1c, 3.1 g, 3.1n and 3.1r are moderately active with low selectivity index. Unfortunately, the other compounds are not active against MESR-CoV.

Interestingly, 6-substituted derivatives 3.1c and 3.1e-j showed low micromolar activity (EC₅₀=1-10 μM) against measles virus, but only compound 3.1c (EC₅₀=0.97 μM) has a high selectivity index (SI₅₀>100). 6,7-Disubstituted analogues 3.1l-r exhibited no activity against measles virus, tacaribe virus and yellow fever virus. 6-Cyclopropylethynyl analogue 3.1c also showed selective submicromolar activity against tacaribe virus and yellow fever virus (anti-tacaribe virus, EC₅₀=0.43 μM and SI₅₀>230; Anti-yellow fever virus, EC₅₀=1.8 μM; SI₅₀=33). Moreover, 6-cyclopentylethynyl 3.1g displayed activity (EC₅₀=0.23 μM) against tacaribe virus, but its cytotoxicity (CC₅₀=34 μM) is somewhat higher than that of 3.1g (CC₅₀>100 μM). It seems that the presence of a terminal substituted ethynyl group increases cytotoxicity in Vero cell. However, the introduction of a cyclic alkyl substituted ethynyl group results in compounds with good activity and low cytotoxicity. Besides compound 3.1c, 6-arylethynyl analogues (3.1h-J) also performed micromolar activity with low selectivity in the anti-yellow fever virus test.

TABLE 1
Antiviral activity against MERS-CoV, measles virus, tacaribe virus and yellow fever virus.

| Drug | | |
|---|---|---|
| Assay | Tacaribe | Yellow |
| name | virus | fever virus |

| | | | | |
|---|---|---|---|---|
| (Cyto- | MERS-CoV | Measles | EC₅₀ | EC₅₀ |

| | pathic | EC₅₀ | CC₅₀ | | EC₅₀ | CC₅₀ | | (μM)^{a} | | | (μM)^{a} | | |
|---|---|---|---|---|---|---|---|---|---|---|---|---|---|
| | effect/ | (μM)^{a} | (μM)^{b} | | (μM)^{a} | (μM)^{b} | | TRVL | CC₅₀ | | YFV | CC₅₀ | |
| Com- | Tox- | EMC | Vero | | CC | Vero | | 11573 | (μM)^{b} | | 17D | (μM)^{b} | |
| pound | icity) | strain | 76 | SI₅₀^{c} | strain | 76 | SI₅₀^{c} | strain | Vero | SI₅₀^{c} | strain | Huh7 | SI₅₀^{c} |
| 3.1a | Visual | >100 | >100 | 0 | 63 | 100 | >1.6 | 100 | 100 | 0 | >100 | 100 | 0 |
| | Neutral | >100 | >100 | 0 | 56 | >100 | >1.8 | >100 | >100 | 0 | >100 | >100 | 0 |
| | Red | | | | | | | | | | | | |
| 3.1b | Visual | 15 | 52 | 3.5 | >100 | >100 | 0 | 44 | 100 | 2.3 | >28 | 28 | 0 |
| | Neutral | 5.8 | 86 | 15 | 64 | 100 | >1.6 | 39 | 99 | 2.5 | >22 | 22 | 0 |
| | Red | | | | | | | | | | | | |
| 3.1c | Visual | 10 | 100 | >10 | 1 | >100 | >100 | 0.68 | >100 | 150 | 0.86 | 32 | 37 |
| | Neutral | 6.7 | >100 | >15 | 0.97 | >100 | >100 | 0.43 | >100 | >230 | 1.8 | 60 | 33 |
| | Red | | | | | | | | | | | | |
| 3.1d | Visual | >3.2 | 3.2 | 0 | >3.6 | 3.6 | 0 | 3.2 | 5.2 | 1.6 | 2.9 | 12 | 4.1 |
| | Neutral | 1.4 | 3.3 | 2.4 | >3.4 | 3.4 | 0 | 2.2 | 4.2 | 1.9 | >1.8 | 1.8 | 0 |
| | Red | | | | | | | | | | | | |
| 3.1e | Visual | 11 | >100 | >9.1 | 3.2 | 34 | 11 | 1.3 | 10 | 7.7 | 9.2 | 28 | 3 |
| | Neutral | 14 | >100 | >7.1 | 2.4 | 52 | 22 | 1 | 7.3 | 7.3 | 9.2 | 28 | 3 |
| | Red | | | | | | | | | | | | |
| 3.1f | Visual | >19 | 19 | 0 | 3.2 | 63 | 20 | 1.6 | 10 | 6.3 | 7.6 | 28 | 3.7 |
| | Neutral | 64 | 110 | 1.7 | 3.7 | 59 | 16 | 1.8 | 9.9 | 5.5 | >31 | 31 | 0 |
| | Red | | | | | | | | | | | | |
| 3.1g | Visual | 7.7 | 100 | 13 | 3.3 | 32 | 9.7 | 0.37 | 24 | 65 | 0.86 | 8.6 | 10 |
| | Neutral | 2.9 | 49 | 17 | 2.8 | 32 | 11 | 0.32 | 34 | 110 | 1.3 | 6 | 4.6 |
| | Red | | | | | | | | | | | | |
| 3.1h | Visual | >32 | 32 | 0 | 6.2 | 28 | 4.5 | 24 | 24 | 0 | 2.2 | 32 | 15 |
| | Neutral | >29 | 29 | 0 | 7.1 | 31 | 4.4 | >23 | 23 | 0 | 2.3 | 30 | 13 |
| | Red | | | | | | | | | | | | |
| 3.1i | Visual | >32 | 32 | 0 | 3.2 | 24 | 7.5 | >12 | 12 | 0 | 1.2 | 32 | 27 |
| | Neutral | >32 | 32 | 0 | 3.6 | 22 | 6.1 | >11 | 11 | 0 | 0.85 | 30 | 35 |
| | Red | | | | | | | | | | | | |
| 3.1j | Visual | >32 | 32 | 0 | 3.2 | 12 | 3.8 | >8.6 | 8.6 | 0 | 1.2 | 28 | 23 |
| | Neutral | >32 | 32 | 0 | 3.3 | 11 | 3.3 | >6.5 | 6.5 | 0 | 0.8 | 5 | 6.3 |
| | Red | | | | | | | | | | | | |
| 3.1l | Visual | >100 | >100 | 0 | 72 | >100 | >1.4 | >100 | >100 | 0 | >100 | >100 | 0 |
| | Neutral | 49 | >100 | >2 | >100 | >100 | 0 | >100 | >100 | 0 | >92 | 92 | 0 |
| | Red | | | | | | | | | | | | |
| 3.1m | Visual | 22 | >100 | >4.5 | 25 | >100 | >4 | >100 | >100 | 0 | 3.2 | 4.6 | 1.4 |
| | Neutral | 14 | >100 | >7.1 | 43 | >100 | >2.3 | >100 | >100 | 0 | >20 | 20 | 0 |
| | Red | | | | | | | | | | | | |
| 3.1n | Visual | 10 | 100 | >10 | 64 | >100 | >1.6 | >100 | >100 | 0 | >100 | 100 | 0 |
| | Neutral | 5.7 | >100 | >18 | 33 | >100 | >3 | >100 | 100 | 0 | >21 | 21 | 0 |
| | Red | | | | | | | | | | | | |
| 3.1o | Visual | >100 | >100 | 0 | 100 | >100 | 0 | >100 | >100 | 0 | >100 | >100 | 0 |
| | Neutral | 68 | >100 | >1.5 | >100 | >100 | 0 | >100 | >100 | 0 | >85 | 85 | 0 |
| | Red | | | | | | | | | | | | |
| 3.1p | Visual | 100 | >100 | 0 | 100 | >100 | 0 | 100 | >100 | 0 | >100 | >100 | 0 |
| | Neutral | >100 | >100 | 0 | >100 | >100 | 0 | >100 | >100 | 0 | >100 | >100 | 0 |
| | Red | | | | | | | | | | | | |
| 3.1q | Visual | >100 | >100 | 0 | >100 | >100 | 0 | >100 | >100 | 0 | >100 | >100 | 0 |
| | Neutral | >100 | >100 | 0 | >100 | >100 | 0 | >100 | >100 | 0 | >100 | >100 | 0 |
| | Red | | | | | | | | | | | | |
| 3.1r | Visual | 3 | 100 | 33 | 7.7 | 32 | 4.2 | >63 | 63 | 0 | 100 | >100 | 0 |
| | Neutral | 2.5 | 46 | 18 | 9.6 | 42 | 4.4 | 43 | 59 | 1.4 | >1.9 | 1.9 | 0 |
| | Red | | | | | | | | | | | | |
| M128533 | Visual | 1.9^{d} | >100^{d} | >53 | / | / | / | / | / | / | / | / | / |
| | Neutral | 0.6^{d} | >100^{d} | >170 | / | / | / | / | / | / | / | / | / |
| | Red | | | | | | | | | | | | |
| Riba- | Visual | / | / | / | / | / | / | 9.2^{d} | >1000^{d} | >110 | / | / | / |
| virin | Neutral | / | / | / | / | / | / | 7.8^{d} | 970^{d} | 120 | / | / | / |
| | Red | | | | | | | | | | | | |
| 2′- | Visual | / | / | / | 1^{d} | >100^{d} | >100 | / | / | / | / | / | / |
| Fluoro- | Neutral | / | / | / | 0.95^{d} | >100^{d} | >110 | / | / | / | / | / | / |
| 2′-de- | Red | | | | | | | | | | | | |
| oxycy- | | | | | | | | | | | | | |
| tidine | | | | | | | | | | | | | |
| Infergen | Visual | / | / | / | / | / | / | / | / | / | <0.00001^{d} | >0.01^{a} | >1000 |
| | Neutral | / | / | / | / | / | / | / | / | / | <0.00001^{d} | >0.01^{d} | >1000 |
| | Red | | | | | | | | | | | | |

^{a}Effective concentration that reduces viral replication by 50%;
^{b}Cytotoxic concentration reduces cell viability by 50%;
^{c}SI₅₀: CC₅₀/EC₅₀;
^{d}Conc. unit: μg/mL.

TABLE 2
Antiviral activity against influenza A (H1N1) virus

| | | EC₅₀ (μM)^{a} | | |
|---|---|---|---|---|
| | | California/ | CC₅₀ | |
| Com- | | 07/2009 | (μM)^{b} | |
| pounds | Drug Assay Name | strain | MDCK | SI₅₀^{c} |
| 3.1a | Visual (Cytopathic | >100 | >100 | 0 |
| | effect/Toxicity) | | | |
| | Neutral Red (Cytopathic | >100 | >100 | 0 |
| | effect/Toxicity) | | | |
| 3.1b | Visual (Cytopathic | >68 | 68 | 0 |
| | effect/Toxicity) | | | |
| | Neutral Red (Cytopathic | >15 | 15 | 0 |
| | effect/Toxicity) | | | |
| 3.1c | Visual (Cytopathic | 11 | >100 | >9.1 |
| | effect/Toxicity) | | | |
| | Neutral Red (Cytopathic | 8.2 | >100 | >12 |
| | effect/Toxicity) | | | |
| 3.1d | Visual (Cytopathic | 3.2 | 6.1 | 1.9 |
| | effect/Toxicity) | | | |
| | Neutral Red (Cytopathic | 2.9 | 4.9 | 1.7 |
| | effect/Toxicity) | | | |
| 3.1e | Visual (Cytopathic | 3.2 | 83 | 26 |
| | effect/Toxicity) | | | |
| | Neutral Red (Cytopathic | 3 | 36 | 12 |
| | effect/Toxicity) | | | |
| 3.1f | Visual (Cytopathic | 2.2 | 9.1 | 4.1 |
| | effect/Toxicity) | | | |
| | Neutral Red (Cytopathic | 2.3 | 5.1 | 2.2 |
| | effect/Toxicity) | | | |
| 3.1g | Visual (Cytopathic | >8.4 | 8.4 | 0 |
| | effect/Toxicity) | | | |
| | Neutral Red (Cytopathic | >7.7 | 7.7 | 0 |
| | effect/Toxicity) | | | |
| 3.1h | Visual (Cytopathic | >32 | 32 | 0 |
| | effect/Toxicity) | | | |
| | Neutral Red (Cytopathic | >14 | 14 | 0 |
| | effect/Toxicity) | | | |
| 3.1i | Visual (Cytopathic | >24 | 24 | 0 |
| | effect/Toxicity) | | | |
| | Neutral Red (Cytopathic | >9.8 | 9.8 | 0 |
| | effect/Toxicity) | | | |
| 3.1j | Visual (Cytopathic | >2.4 | 2.4 | 0 |
| | effect/Toxicity) | | | |
| | Neutral Red (Cytopathic | >3.2 | 3.2 | 0 |
| | effect/Toxicity) | | | |
| 3.1l | Visual (Cytopathic | 0.52 | >100 | >190 |
| | effect/Toxicity) | | | |
| | Neutral Red (Cytopathic | 0.34 | >100 | >290 |
| | effect/Toxicity) | | | |
| 3.1m | Visual (Cytopathic | >100 | >100 | 0 |
| | effect/Toxicity) | | | |
| | Neutral Red (Cytopathic | >100 | >100 | 0 |
| | effect/Toxicity) | | | |
| 3.1n | Visual (Cytopathic | >100 | >100 | 0 |
| | effect/Toxicity) | | | |
| | Neutral Red (Cytopathic | >100 | >100 | 0 |
| | effect/Toxicity) | | | |
| 3.1o | Visual (Cytopathic | >100 | >100 | 0 |
| | effect/Toxicity) | | | |
| | Neutral Red (Cytopathic | >100 | >100 | 0 |
| | effect/Toxicity) | | | |
| 3.1p | Visual (Cytopathic | >100 | >100 | 0 |
| | effect/Toxicity) | | | |
| | Neutral Red (Cytopathic | >100 | >100 | 0 |
| | effect/Toxicity) | | | |
| 3.1q | Visual (Cytopathic | >100 | >100 | 0 |
| | effect/Toxicity) | | | |
| | Neutral Red (Cytopathic | >100 | >100 | 0 |
| | effect/Toxicity) | | | |
| 3.1r | Visual (Cytopathic | 0.32 | 32 | 100 |
| | effect/Toxicity) | | | |
| | Neutral Red (Cytopathic | 0.34 | 43 | 130 |
| | effect/Toxicity) | | | |
| Ribavirin | Visual (Cytopathic | 2.2^{d} | >1000^{d} | >450 |
| | effect/Toxicity) | | | |
| | Neutral Red (Cytopathic | 2.4^{d} | >1000^{d} | >420 |
| | effect/Toxicity) | | | |

^{a}Effective concentration that reduces viral replication by 50%;
^{b}Cytotoxic concentration that reduces cell viability by 50%;
^{c}SI₅₀: CC₅₀/EC₅₀;
^{d}Concentration unit: μg/mL.

### 3.2. Antiviral Activity Against Influenza a (H1N1) Virus

Antiviral activity and cytotoxicity of reference compound 3.1a and compounds of the invention 3.1b-j and 3.11-r was tested against influenza A (H1N1) virus (California/01/2009 strain) in MDCK cell line (Table 2) and ribavirin was used as positive control. Interestingly, 3.1c with cyclopropylethynyl substituted at the 6-position showed moderate activity, but reference compound 3.1a was not active. It indicated that the ethynyl group is necessary for the antiviral activity. In the further screening, 6-alkylethynyl 3.1d-f displayed micromolar activity and low selectivity. However, other derivatives 3.1g-j exhibited no anti-influenza A activity. Among 6,7-disubstituted 7-deazaadenosine analogues, 7-ethynyl-6-ethyl-7-deazaadenosine (3.11) exhibited significant activity (EC₅₀=0.34 μM) with no cytotoxicity as well as high selectivity (SI₅₀>290). In addition, 7-fluoro-6-ethyl 3.1r had similar activity with compound 3.11. The other compounds showed no activity against influenza A virus. In conclusion, the 6-ethyl compound remained the most active and selective anti-influenza A compound and an ethynyl group and a fluorine atom at the 7-position are tolerated.

### 3.3. Antiviral Activity Against Human Norovirus

Reference compound 3.1a and 6-Substituted 7-deazaadenosine (3.1b-h, 3.1j (Scheme 2)) and 6,7-disubstituted analogues (3.11-n) were evaluated for their antiviral activity and cytotoxicity against human norovirus genogroup I NOV in HG23 Nov replicon cells in quantitative reverse transcription polymerase chain reaction (qRT PCR) (intracellular RNA)/β-Actin (toxicity) assays, while 2′C methyl cytidine was used as positive control.

As shown in Table 3, the 6-substituted analogues of the invention showed submicromolar activity to nanomolar activity against human norovirus, specially compounds 3.1b, 3.1d, 3.1f, and 3.1h with excellent selectivity. Although reference compound 3.1a exhibited almost no cytotoxicity, its micromolar activity (EC₅₀=2.52 μM) and selectivity (SI₅₀>40) was much weaker than that of other 6-substituted analogues. Compound 3.1b displayed potent antiviral activity (EC₅₀=0.007 μM) and is less cytotoxic than other alkyl- and arylethynyl analogues. Among this series of nonterminal ethynyl substituted derivatives, compounds 3.1d and 3.1f (EC₅₀s of 0.006 μM and 0.002 μM, respectively) with small and flexible substituents were more active than analogues (3.1c, 3.1e, and 3.1g) with the cyclic and branched substituents. Moreover, significant antiviral activity of analogues 3.1h (EC₅₀<0.001 μM, SI₅₀>38,470) and 3.1j (EC₅₀=0.022 μM, SI₅₀=1,748) indicated that the phenylethynyl group at the 6-position contributed to the potent anti-norovirus activity and that a fluorine atom at the 4-position of the phenyl group is more effective than a chlorine atom. However, 6,7-disubstituted analogues 3.11, 3.1m and 3.1n showed no promising activity because of their significant cytotoxicity.

TABLE 3
Activity against human norovirus genogroup
NoV in HG23 Nov replicon cells

| Compound | EC₅₀ (μM)^{a} | CC₅₀ (μM)^{b} | SI₅₀^{c} |
|---|---|---|---|
| 3.1a | 2.52 | >100 | >40 |
| 3.1b | 0.007 | 60.51 | 8,846 |
| 3.1c | 0.126 | 17.49 | 139 |
| 3.1d | 0.006 | 17.36 | 3,145 |
| 3.1e | 0.396 | 39.47 | 100 |
| 3.1f | 0.002 | 38.72 | 16,762 |
| 3.1g | 0.438 | 25.79 | 59 |
| 3.1h | <0.001 | 38.47 | >38,470 |
| 3.1j | 0.022 | 38.64 | 1,748 |
| 3.1l | 0.393 | 0.455 | 1 |
| 3.1m | 3.35 | 14.61 | 4 |
| 3.1n | 0.062 | 0.342 | 6 |
| 2′C Methyl Cytidine | 1.57 | >25.00 | >16 |

^{a}Effective concentration that reduces viral replication by 50%;
^{b}Cytotoxic concentration that reduces cell viability by 50%;
^{c}SI₅₀: CC₅₀/EC₅₀.

In general, the introduction of the alkynyl groups enhanced antiviral activity and were more cytotoxic than 6-alkyl/aryl derivatives, but sometimes 6-alkylnyl analogues showed higher selectivity index. For example, compound 3.1f with nanomolar activity showed a SI₅₀ of 16762, which was higher than derivative 3.2c. Interestingly, 6-cyclopentylethynyl 3.1g showed submicromolar activity (EC₅₀=0.438 μM) with low selectivity index (SI₅₀=59). Compound 3.1h with the (4-fluorophenyl)ethynyl group had good activity with an excellent selectivity index (SI₅₀>38,470). The 6-substituted 7-deazaadenosine analogues of the invention displayed exciting antiviral activity against human norovirus and were much more active than the positive control (2′-C-Methylcytidine)

### Example 4. Experimental Section

All reagents and solvents were of analytical grade and used without further purification. All sensitive reactions were carried out in dry solvents under an argon or nitrogen atmosphere. ¹H, ¹³C, and ³¹P NMR spectra were obtained on a 300, 500 or 600 MHz Bruker Avance spectrometer using tetramethylsilane (TMS) as an internal standard or by referencing to the residual solvent signal. Coupling constants are reported in hertz (Hz) and were directly obtained from the spectra. The following abbreviations were used to denote the NMR splitting patterns: s (singlet), d (doublet), t (triplet), q (quartet), dd (doublet of doublets), dt (doublet of triplets), m (multiplet), and br (broad). High-resolution mass spectra (HRMS) were obtained on a quadrupole orthogonal acceleration time-of-flight mass spectrometer (Synapt G2, HDMS, Waters, Milford, MA). Samples were infused at 3 μL/min, and spectra were obtained in positive (or negative) ionization mode with a resolution of 15 000 FWHM using leucine enkephalin as the lock mass. Pre-coated aluminum sheets (254 nm) were used for thin layer chromatography (TLC). Intermediate compounds were purified by silica gel column chromatography (60 Å, 0.060-0.200 mm, Acros Organics). Purities of all of the tested compounds were above 95% by HPLC analysis.

2′,3′,5′-Tri-O-benzoyl-6-chloro-9-β-D-ribofuranosyl-7-deazapurine (3.7). To a solution of 3.6 (5.20 g, 7.18 mmol) in anhydrous THE (30 mL) was added dropwise 1.3 M iPrMgCl·LiCl in THE (5.80 mL) at −10° C. The mixture was stirred for 30 min at the same temperature. The reaction was poured into ice and sat. aqueous NH₄Cl and extracted with EtOAc. The organic layer was dried over MgSO₄, filtered and concentrated in vacuo. The residue was purified by column chromatography (silica gel, petroleum ether/EtOAc=5/1) to afford 3.7 (3.55 g, 82% yield) as a yellow foam.

¹H NMR (300 MHz, CDCl₃): δ 8.60 (1H, s, H-2), 8.11 (2H, d, J=7.2 Hz, Ph-H), 8.01 (2H, d, J=7.2 Hz, Ph-H), 7.93 (2H, d, J=7.9 Hz, Ph-H), 7.64-7.33 (10H, m, Ph-H and H-8), 6.69 (1H, d, J=5.5 Hz, H-1′), 6.62 (1H, d, J=3.8 Hz, H-7), 6.25 (1H, t, J=5.7 Hz, H-2′), 6.16 (1H, t, J=5.1 Hz, H-3′), 4.89 (1H, dd, J₁=12.1 Hz, J2=3.1 Hz, H-5′a), 4.81 (1H, q, J=3.8 Hz, H-4′), 4.69 (1H, dd, J₁=12.1 Hz, J₂=3.8 Hz, H-5′b). ¹³C NMR (75 MHz, CDCl₃): δ 166.2 (COPh), 165.5 (COPh), 165.2 (COPh), 152.7 (C-6), 151.6 (C-4), 151.3 (C-2), 133.9 (Ph-C), 133.6 (Ph-C), 129.9 (Ph-C), 129.8 (Ph-C), 129.5 (Ph-C), 128.8 (Ph-C), 128.8 (Ph-C), 128.7 (Ph-C), 128.6 (Ph-C), 128.5 (Ph-C), 126.8 (C-8), 118.7 (C-5), 101.6 (C-7), 86.9 (C-1′), 80.5 (C-4′), 74.1 (C-2′), 71.6 (C-3′), 63.8 (C-5′). HRMS (ESI⁺): m/z, [M+H]⁺ calcd for C₃₂H₂₅ClN₃O₇, 598.1375; found, 598.1375.

2′,3′,5′-Tri-O-benzoyl-6-ethyl-9-β-D-ribofuranosyl-7-deazapurine (3.9). To a solution of 3.7 (500 mg, 0.836 mmol), Fe(acac)3 (30 mg, 0.084 mmol), CuI (32 mg, 0.167 mmol) and NMP (2.0 mL) in anhydrous THE (20 mL) was added dropwise 3 M EthylMgBr in THE (0.61 mL) at 0° C. and the reaction mixture was stirred for 45 min at the same temperature. The reaction was quenched with sat. aqueous NH₄Cl and extracted with EtOAc. The organic layer was dried over MgSO₄, filtered and concentrated in vacuo. The residue was purified by column chromatography (silica gel, petroleum ether/EtOAc=5/1, 3/1) to afford 3.9 (400 mg, 80% yield) as a white solid.

¹H NMR (300 MHz, CDCl₃): δ 8.81 (1H, s, H-2), 8.16-8.12 (2H, m, Ph-H), 8.02-7.98 (2H, m, Ph-H), 7.96-7.92 (2H, m, Ph-H), 7.63-7.31 (10H, m, Ph-H and H-8), 6.77 (1H, d, J=5.8 Hz, H-1′), 6.59 (1H, d, J=3.8 Hz, H-7), 6.29 (1H, t, J=5.8 Hz, H-2′), 6.19 (1H, dd, J₁=5.8 Hz, J₂=4.3 Hz, H-3′), 4.88 (1H, dd, J₁=12.0 Hz, J2=3.1 Hz, H-5′a), 4.80 (1H, q, J=3.8 Hz, H-4′), 4.70 (1H, dd, J₁=12.3 Hz, J₂=3.9 Hz, H-5′b), 3.01 (2H, q, J=7.6 Hz, CH₂), 1.38 (3H, t, J=7.6 Hz, CH₃). 13C NMR (75 MHz, CDCl₃): 5166.2 (COPh), 165.5 (COPh), 165.2 (COPh), 164.7 (C-6), 151.9 (C-2), 151.1 (C-4), 133.7 (Ph-C), 133.4 (Ph-C), 129.9 (Ph-C), 129.8 (Ph-C), 129.5 (Ph-C), 128.8 (Ph-C), 128.7 (Ph-C), 128.6 (Ph-C), 128.6 (Ph-C), 128.5 (Ph-C), 125.0 (C-8), 118.0 (C-5), 101.3 (C-7), 86.2 (C-1′), 80.2 (C-4′), 73.9 (C-2′), 71.6 (C-3′), 64.0 (C-5′), 28.6 (CH₂), 12.8 (CH₃).

2′,3′,5′-Tri-O-benzoyl-6-ethyl-7-iodo-9-β-D-ribofuranosyl-7-deazapurine (3.10). To a mixture of 3.9 (400 mg, 0.676 mmol) in anhydrous DMF (8 mL) was added dropwise NIS (182 mg, 0.811 mmol) in anhydrous DMF (2 mL) and stirred at room temperature overnight. To the mixture was added water and EtOAc. The aqueous layer was extracted with EtOAc and the combined organic layer was washed with water, brine, dried over MgSO₄, filtered and concentrated in vacuo. The crude product was purified by column chromatography (silica gel, heptane/EtOAc=3/1, 2/1) to afford 3.10 (245 mg, 50% yield) as a yellow foam.

¹H NMR (300 MHz, CDCl₃): δ 8.77 (1H, s, H-2), 8.16-8.11 (2H, m, Ph-H), 8.01-7.92 (4H, m, Ph-H), 7.64-7.30 (10H, m, Ph-H and H-8), 6.73 (1H, d, J=5.3 Hz, H-1′), 6.20-6.11 (2H, m, H-2′ and H-3′), 4.89 (1H, dd, J₁=12.1 Hz, J₂=3.1 Hz, H-5′a), 4.79 (1H, q, J=3.6 Hz, H-4′), 4.69 (1H, dd, J₁=12.1 Hz, J₂=3.6 Hz, H-5′a), 3.31 (2H, q, J=7.5 Hz, CH₂), 1.37 (3H, t, J=7.5 Hz, CH₃). ¹³C NMR (75 MHz, CDCl₃): δ 166.3 (COPh), 165.9 (C-6), 165.5 (COPh), 165.2 (COPh), 152.1 (C-2), 150.7 (C-4), 133.8 (Ph-C), 133.6 (Ph-C), 130.5 (Ph-C), 130.0 (Ph-C), 129.9 (Ph-C), 129.9 (Ph-C), 129.5 (Ph-C), 128.9 (Ph-C), 128.8 (Ph-C), 128.6 (Ph-C), 128.6 (C-8), 118.1 (C-5), 86.3 (C-1′), 80.5 (C-4′), 74.2 (C-2′), 71.6 (C-3′), 63.8 (C-5′), 53.8 (C-7), 26.7 (CH₂), 14.0 (CH₃). HRMS (ESI⁺): m/z, [M+H]⁺ calcd for C₃₄H₂₉I₁N₃O₇, 718.1046; found, 718.1067.

6-Cyclopropyl-9-β-D-ribofuranosyl-7-deazapurine (3.1a). To a solution of 3.7 (130 mg, 4.89 mmol), Fe(acac)3 (8 mg, 0.023 mmol), CuI (9 mg, 0.044 mmol) and NMP (0.50 mL) in anhydrous THE (5 mL) was added dropwise 0.5-0.7 M CyclopropylMgBr in THE (1.10 mL) at 0° C. and the reaction mixture was stirred for 45 min at room temperature. The reaction was quenched with sat. aqueous NH₄Cl and extracted with EtOAc. The organic layer was dried over MgSO₄, filtered and concentrated in vacuo. The residue was purified by column chromatography (silica gel, petroleum ether/EtOAc=5/1, 3/1) to afford 3.8a (90 mg) which was dissolved in 7 M NH₃ in MeOH (5 mL) and stirred overnight at room temperature. The mixture was concentrated in vacuo. The residue was purified by column chromatography (silica gel, CH₂Cl₂/MeOH=20/1) to afford 3.1a (42 mg, 65% yield, over two steps) as a yellow solid.

¹H NMR (300 MHz, MeOD): δ 8.51 (1H, s, H-2), 7.63 (1H, d, J=3.8 Hz, H-8), 6.79 (1H, d, J=3.8 Hz, H-7), 6.17 (1H, d, J=6.3 Hz, H-1′), 4.64 (1H, t, J=5.8 Hz, H-2′), 4.31 (1H, dd, J₁=5.3 Hz, J₂=3.0 Hz, H-3′), 4.12 (1H, q, J=3.0 Hz, H-4′), 3.86 (1H, dd, J₁=12.3 Hz, J₂=2.9 Hz, H-5′a), 3.75 (1H, dd, J₁=12.3 Hz, J2=3.2 Hz, H-5′b), 2.49-2.40 (1H, m, CH), 1.30-1.24 (2H, m, CH₂), 1.23-1.14 (2H, m, CH₂). ¹³C NMR (75 MHz, MeOD): δ 166.2 (C-6), 151.6 (C-2), 150.7 (C-4), 128.3 (C-8), 119.8 (C-5), 100.9 (C-7), 90.6 (C-1′), 87.1 (C-4′), 75.5 (C-2′), 72.5 (C-3′), 63.4 (C-5′), 15.2 (CH), 11.5 ((CH₂)₂). HRMS (ESI⁺): m/z, [M+H]⁺ calcd for C₁₄H₁₈N₃O₄⁺, 292.1291; found, 292.1293.

6-(1-Ethoxyvinyl)-9-β-D-ribofuranosyl-7-deazapurine (3.1b). To a N₂-purged solution of 3.7 (100 mg, 0.167 mmol) and Pd(PPh₃)₂Cl₂ (5.8 mg, 0.008 mmol) in anhydrous DMF (3 mL) was added tributyl(1-ethoxyvinyl)tin (67 μL, 0.200 mmol) and the reaction mixture was stirred for 15 h at 100° C. The mixture was concentrated in vacuo. The residue was purified by column chromatography (silica gel, petroleum ether/EtOAc=5/1, 3/1) to afford crude 3.8b (86 mg) which was dissolved in 7 M NH₃ in MeOH (5 mL) and stirred at r.t overnight. The mixture was concentrated in vacuo. The residue was purified by column chromatography (silica gel, CH₂Cl₂/MeOH=20/1) to afford 3.1b (35 mg, 65% yield, over two steps) as a white solid.

¹H NMR (300 MHz, MeOD): δ 8.67 (1H, s, H-2), 7.71 (1H, d, J=3.8 Hz, H-8), 6.97 (1H, d, J=3.8 Hz, H-7), 6.25 (1H, d, J=6.1 Hz, H-1′), 5.56 (1H, d, J=2.3 Hz, vinyl-CH₂a), 4.67 (1H, d, J=2.4 Hz, vinyl-CH₂b), 4.62 (1H, t, J=5.7 Hz, H-2′), 4.31 (1H, dd, J₁=5.3 Hz, J₂=3.2 Hz, H-3′), 4.12 (1H, q, J=3.2 Hz, H-4′), 4.05 (2H, q, J=6.9 Hz, CH₂), 3.86 (1H, dd, J₁=12.2 Hz, J₂=3.0 Hz, H-5′a), 3.76 (1H, dd, J₁=12.3 Hz, J₂=3.4 Hz, H-5′b), 1.51 (3H, t, J=7.0 Hz, CH₃). ¹³C NMR (75 MHz, MeOD): δ 160.2 (vinyl-C), 154.0 (C-6), 153.7 (C-4), 150.9 (C-2), 129.2 (C-8), 117.4 (C-5), 104.3 (C-7), 90.2 (C-1′), 89.5 (vinyl-CH₂), 86.9 (C-4′), 75.6 (C-3′), 72.4 (C-2′), 65.1 (CH₂), 63.3 (C-5′), 14.9 (CH₃). HRMS (ESI⁺): m/z, [M+H]⁺ calcd for C₁₅H₂₀N₃O₅⁺, 322.1397; found, 322.1397.

### Synthesis of Derivatives 3.1c-j Via Cross-Coupling Reaction and Deprotection.

To a N2-purged solution of 3.7 (0.167 mmol, 1 equiv.), aryl-/alley-acetylenes (10 equiv.), 5% Pd(PPh₃)₂C12 (5.8 mg) and 10% CuI (3.2 mg) in anhydrous DMF (3 mL) was added Et₃N (3 equiv.) and stirred for 15 h at 50° C. The mixture was concentrated in vacuo and the residue was purified by column chromatography (silica gel, petroleum ester/EtOAc=5/1, 3/1) to give a series of crude intermediate 3.8 (3.8c-j, Scheme 2). The crude compound was treated with 7M NH₃ in MeOH (5 mL) and stirred at room temperature overnight. After removal of the solvent, the residue was purified with the column chromatography (silica gel, CH₂Cl₂/MeOH=20/1, 15/1) to give the desired ribonucleoside analogue.

6-Cydopropylethynyl-9-β-D-ribofuranosyl-7-deazapurine (3.1c). As described in the general procedure, compound 3.1c was afforded as a yellow solid (40 mg, 75% yield, over two steps) from 3.7 (100 mg, 0.167 mmol), cyclopropylacetylene (0.14 mL, 1.67 mmol), Pd(PPh₃)Cl₂ (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.62 (1H, s, H-2), 7.78 (1H, d, J=3.8 Hz, H-8), 6.66 (1H, d, J=3.7 Hz, H-7), 6.23 (1H, d, J=6.1 Hz, H-1′), 4.60 (1H, t, J=5.7 Hz, H-2′), 4.31 (1H, dd, J₁=5.3 Hz, J₂=3.3 Hz, H-3′), 4.11 (1H, q, J=3.2 Hz, H-4′), 3.85 (1H, dd, J₁=12.2 Hz, J₂=3.0 Hz, H-5′a), 3.75 (1H, dd, J₁=12.3 Hz, J₂=3.4 Hz, H-5′b), 1.70-1.61 (1H, m, CH), 1.08-1.00 (2H, m, CH₂), 0.98-0.91 (2H, m, CH₂). ¹³C NMR (75 MHz, MeOD): δ 151.9 (C-4), 151.5 (C-2), 143.6 (C-6), 129.9 (C-8), 122.2 (C-5), 104.0 (alkynyl-C), 101.6 (C-7), 90.1 (C-1′), 87.0 (C-4′), 75.8 (C-2′), 73.1 (alkynyl-C), 72.3 (C-3′), 63.2 (C-5′), 9.9 ((CH₂)₂), 0.9 (CH). HRMS (ESI⁺): m/z, [M+H]⁺ calcd for C₁₆H₁₈N₃O₄⁺, 316.1291; found, 316.1290.

6-(Prop-1-yn-1-yl)-9-β-D-ribofuranosyl-7-deazapurine (3.1d). As described in the general procedure, compound 3.1d was afforded as a white solid (25 mg, 51% yield, over two steps) from 3.7 (100 mg, 0.167 mmol), 1M 1-propyne in DMF (1.67 mL, 1.67 mmol), Pd(PPh₃)₂Cl₂ (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.65 (1H, s, H-2), 7.80 (1H, d, J=3.7 Hz, H-8), 6.70 (1H, d, J=3.7 Hz, H-7), 6.24 (1H, d, J=6.0 Hz, H-1′), 4.60 (1H, t, J=5.6 Hz, H-2′), 4.31 (1H, dd, J₁=5.3 Hz, J₂=3.3 Hz, H-3′), 4.11 (1H, q, J=3.2 Hz, H-4′), 3.85 (1H, dd, J₁=12.2 Hz, J₂=3.0 Hz, H-5′a), 3.75 (1H, dd, J₁=12.2 Hz, J₂=3.4 Hz, H-5′b), 2.22 (3H, s, CH₃). ¹³C NMR (75 MHz, MeOD): δ 152.1 (C-4), 151.5 (C-2), 143.7 (C-6), 130.0 (C-8), 122.4 (C-5), 101.6 (C-7), 96.1 (alkynyl-C), 90.1 (C-1′), 87.0 (C-4′), 77.0 (alkynyl-C), 75.8 (C-2′), 72.3 (C-3′), 63.2 (C-5′), 4.1 (CH₃). HRMS (ESI⁺): m/z, [M+H]⁺ calcd for C₁₄H₁₆N₃O₄⁺, 290.1135; found, 290.1137.

6-(3-Methylbut-1-yn-1-yl)-9-β-D-ribofuranosyl-7-deazapurine (3.1e). As described in the general procedure, compound 3.1e was afforded as a white solid (37 mg, 69% yield, over two steps) from 3.7 (100 mg, 0.167 mmol), 3-methyl-1-butyne (0.16 mL, 1.67 mmol), Pd(PPh₃)₂Cl₂ (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.66 (1H, s, H-2), 7.81 (1H, d, J=3.7 Hz, H-8), 6.68 (1H, d, J=3.7 Hz, H-7), 6.24 (1H, d, J=6.0 Hz, H-1′), 4.60 (1H, t, J=5.7 Hz, H-2′), 4.31 (1H, dd, J₁=5.3 Hz, J₂=3.3 Hz, H-3′), 4.11 (1H, q, J=3.2 Hz, H-4′), 3.86 (1H, dd, J₁=12.2 Hz, J₂=2.7 Hz, H-5′a), 3.76 (1H, dd, J₁=12.4 Hz, J₂=3.2 Hz, H-5′b), 3.01-2.91 (1H, m, CH), 1.35 (6H, d, J=6.9 Hz, (CH₃)₂). ¹³C NMR (75 MHz, MeOD): δ 152.1 (C-4), 151.5 (C-2), 143.6 (C-6), 130.0 (C-8), 122.3 (C-5), 105.3 (alkynyl-C), 101.6 (C-7), 90.1 (C-1′), 87.0 (C-4′), 77.3 (alkynyl-C), 75.8 (C-2′), 72.3 (C-3′), 63.2 (C-5′), 22.8 ((CH₃)₂), 22.5 (CH). HRMS (ESI⁺): m/z, [M+H]⁺ calcd for C₁₆H₂₀N₃O₄, 318.1448; found, 318.1453.

6-(Pent-1-yn-1-yl)-9-β-D-ribofuranosyl-7-deazapurine (3.1f). As described in the general procedure, compound 3.1f was afforded as a white solid (36 mg, 67% yield, over two steps) from 3.7 (100 mg, 0.167 mmol), 1-pentyne (0.16 mL, 1.67 mmol), Pd(PPh₃)₂C12 (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.66 (1H, s, H-2), 7.81 (1H, d, J=3.8 Hz, H-8), 6.69 (1H, d, J=3.7 Hz, H-7), 6.24 (1H, d, J=6.0 Hz, H-1′), 4.60 (1H, t, J=5.6 Hz, H-2′), 4.31 (1H, dd, J₁=5.3 Hz, J₂=3.3 Hz, H-3′), 4.11 (1H, q, J=3.2 Hz, H-4′), 3.86 (1H, dd, J₁=12.2 Hz, J₂=3.0 Hz, H-5′a), 3.76 (1H, dd, J₁=12.2 Hz, J₂=3.4 Hz, H-5′b), 2.58 (2H, t, J=7.0 Hz, CH₂), 1.79-1.66 (2H, m, CH₂), 1.12 (3H, t, J=7.4 Hz, CH₃). ¹³C NMR (75 MHz, MeOD): δ 152.1 (C-4), 151.5 (C-2), 143.7 (C-6), 130.0 (C-8), 122.4 (C-5), 101.6 (C-7), 100.2 (alkynyl-C), 90.1 (C-1′), 87.0 (C-4′), 78.2 (alkynyl-C), 75.8 (C-2′), 72.3 (C-3′), 63.2 (C-5′), 22.8 (CH₂), 22.1 (CH₂), 13.8 (CH₃). HRMS (ESI⁺): m/z, [M+H]⁺ calcd for C₁₆H₂₀N₃O₄⁺, 318.1448; found, 318.1443.

6-(Cydopentylethynyl)-9-β-D-ribofuranosyl-7-deazapurine (3.1g). As described in the general procedure, compound 3.1g was afforded as a white solid (40 mg, 69% yield, over two steps) from 3.7 (100 mg, 0.167 mmol), cyclopentylacetylene (0.19 mL, 1.67 mmol), Pd(PPh₃)₂C12 (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.65 (1H, s, H-2), 7.80 (1H, d, J=3.7 Hz, H-8), 6.68 (1H, d, J=3.7 Hz, H-7), 6.24 (1H, d, J=6.1 Hz, H-1′), 4.60 (1H, t, J=5.7 Hz, H-2′), 4.31 (1H, dd, J₁=5.3 Hz, J₂=3.3 Hz, H-3′), 4.11 (1H, q, J=3.2 Hz, H-4′), 3.85 (1H, dd, J₁=12.2 Hz, J₂=3.0 Hz, H-5′a), 3.75 (1H, dd, J₁=12.2 Hz, J₂=3.4 Hz, H-5′b), 3.09-2.98 (1H, m, CH), 2.15-2.05 (2H, m, CH₂), 1.85-1.66 (6H, m, (CH₂)₃). ¹³C NMR (75 MHz, MeOD): δ 152.1 (C-4), 151.5 (C-2), 143.8 (C-6), 130.0 (C-8), 122.3 (C-5), 104.5 (alkynyl-C), 101.6 (C-7), 90.1 (C-1′), 87.0 (C-4′), 77.6 (alkynyl-C), 75.8 (C-2′), 72.4 (C-3′), 63.3 (C-5′), 34.7 ((CH₂)₂), 31.9 (CH), 26.1 ((CH₂)₂). HRMS (ESI⁺): m/z, [M+H]⁺ calcd for C₁₈H₂₂N₃O₄⁺, 344.1604; found, 344.1602.

6-((4-Fluorophenyl)ethynyl)-9-β-D-ribofuranosyl-7-deazapurine (3.1h). As described in the general procedure, compound 3.1h was afforded as a yellow solid (46 mg, 74% yield, over two steps) from 3.7 (100 mg, 0.167 mmol), 4-fluorophenylacetylene (0.19 mL, 1.67 mmol), Pd(PPh₃)₂Cl₂ (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.72 (1H, s, H-2), 7.87 (1H, d, J=3.8 Hz, H-8), 7.73 (2H, dd, J₁=8.7 Hz, J₂=5.5 Hz, Ar—H), 7.21 (2H, t, J=8.8 Hz, Ar—H), 6.82 (1H, d, J=3.7 Hz, H-7), 6.28 (1H, d, J=5.9 Hz, H-1′), 4.62 (1H, t, J=5.6 Hz, H-2′), 4.33 (1H, dd, J₁=5.3 Hz, J₂=3.3 Hz, H-3′), 4.13 (1H, q, J=3.3 Hz, H-4′), 3.87 (1H, dd, J₁=12.2 Hz, J₂=3.1 Hz, H-5′a), 3.75 (1H, dd, J₁=12.3 Hz, J₂=3.4 Hz, H-5′b). ¹³C NMR (75 MHz, MeOD): δ 166.7, 163.3 (Ar—C), 152.3 (C-4), 151.7 (C-2), 142.7 (C-6), 135.8, 135.7 (Ar—C), 130.5 (C-8), 122.3 (C-5), 118.8, 118.8 (Ar—C), 117.3, 117.0 (Ar—C), 101.6 (C-7), 96.2 (alkynyl-C), 90.1 (C-1′), 87.0 (C-4′), 85.7 (alkynyl-C), 75.9 (C-2′), 72.3 (C-3′), 63.2 (C-5′). HRMS (ESI⁺): m/z, [M+H]⁺ calcd for C₁₉H₁₇F₁N₃O₄⁺, 370.1197; found, 370.1190.

6-((3-Fluorophenyl)ethynyl)-9-β-D-ribofuranosyl-7-deazapurine (3.11). As described in the general procedure, compound 3.11 was afforded as a yellow solid (39 mg, 63% yield, over two steps) from 3.7 (100 mg, 0.167 mmol), 3-fluorophenylacetylene (0.19 mL, 1.67 mmol), Pd(PPh₃)₂C12 (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (300 MHz, MeOD): δ 8.74 (1H, s, H-2), 7.89 (1H, d, J=3.7 Hz, H-8), 7.54-7.42 (3H, m, Ar—H), 7.29-7.21 (1H, m, Ar—H), 6.83 (1H, d, J=3.7 Hz, H-7), 6.29 (1H, d, J=6.0 Hz, H-1′), 4.62 (1H, t, J=5.6 Hz, H-2′), 4.33 (1H, dd, J₁=5.3 Hz, J₂=3.4 Hz, H-3′), 4.13 (1H, q, J=3.2 Hz, H-4′), 3.87 (1H, dd, J₁=12.2 Hz, J₂=3.0 Hz, H-5′a), 3.77 (1H, dd, J₁=12.2 Hz, J₂=3.4 Hz, H-5′b). ¹³C NMR (75 MHz, MeOD): δ 165.5, 162.2 (Ar—C), 152.3 (C-4), 151.7 (C-2), 142.3 (C-6), 131.9, 131.8 (Ar—C), 130.6 (C-8), 129.5, 129.5 (Ar—C), 124.5, 124.3 (Ar—C), 122.4 (C-5), 120.0, 119.7 (Ar—C), 118.6, 118.3 (Ar—C), 101.6 (C-7), 95.5, 95.5 (alkynyl-C), 90.1 (C-1′), 87.0 (C-4′), 86.5 (alkynyl-C), 75.9 (C-2′), 72.3 (C-3′), 63.2 (C-5′). HRMS (ESI⁺): m/z, [M+H]⁺ calcd for C₁₉H₁₇F₁N₃O₄⁺, 370.1197; found, 370.1195.

6-((4-Chlorophenyl)ethynyl)-9-β-D-ribofuranosyl-7-deazapurine (3.1j). As described in the general procedure, compound 3.1j was afforded as a yellow solid (34 mg, 52% yield, over two steps) from 3.7 (100 mg, 0.167 mmol), (4-chlorophenyl)acetylene (228 mg, 1.67 mmol), Pd(PPh₃)₂C12 (5.8 mg), CuI (3.2 mg) and Et₃N (0.07 mL, 0.501 mmol) in anhydrous DMF (3 mL) and deprotection by treatment with 7 M NH₃ in MeOH (5 mL).

¹H NMR (600 MHz, MeOD): δ 8.74 (1H, s, H-2), 7.88 (1H, d, J=3.8 Hz, H-8), 7.69-7.66 (2H, m, Ar—H), 7.46-7.49 (2H, m, Ar—H), 6.82 (1H, d, J=3.7 Hz, H-7), 6.28 (1H, d, J=6.0 Hz, H-1′), 4.62 (1H, t, J=5.6 Hz, H-2′), 4.33 (1H, dd, J₁=5.3 Hz, J₂=3.4 Hz, H-3′), 4.13 (1H, q, J=3.3 Hz, H-4′), 3.87 (1H, dd, J₁=12.2 Hz, J₂=3.0 Hz, H-5′a), 3.77 (1H, dd, J₁=12.2 Hz, J₂=3.4 Hz, H-5′b). ¹³C NMR (151 MHz, MeOD): δ 152.3 (C-4), 151.7 (C-2), 142.5 (C-6), 137.3 (Ar—C), 134.8 (Ar—C), 130.6 (C-8), 130.2 (Ar—C), 122.4 (C-5), 121.2 (Ar—C), 101.6 (C-7), 95.9 (alkynyl-C), 90.1 (C-1′), 87.0 (C-4′), 86.8 (alkynyl-C), 75.9 (C-2′), 72.3 (C-3′), 63.2 (C-5′). HRMS (ESI⁺): m/z, [M+Na]⁺ calcd for C₁₉H₁₆Cl₁N₃O₄Na⁺, 408.0721; found, 408.0711.

6-Ethyl-7-ethynyl-9-β-D-ribofuranosyl-7-deazapurine (3.11). To a N2-purged solution of 3.10 (50 mg, 0.069 mmol), TES acetylene (0.12 mL, 0.697 mmol), Pd(PPh₃)₂C12 (2.4 mg) and CuI (1.3 mg) in anhydrous DMF (3 mL) was added Et₃N (0.03 mL, 0.209 mmol) and stirred for 15 h at 50° C. The mixture was concentrated and purified by column chromatography (silica gel, petroleum ether/EtOAc=7/1) to afford protected 7-trimethylsilylethynyl intermediate (49 mg) which was dissolved in anhydrous MeOH (5 mL), followed by adding K₂CO₃ (49 mg, 0.355 mmol) and then stirred for 0.5 h at room temperature. The mixture was concentrated and purified by column chromatography (silica gel, CH₂Cl₂/MeOH=20/1) to afford 7-trimethylsilylethynyl-7-deazapurine ribonucleoside analogue. This compound was dissolved in anhydrous THF, followed by adding 1M TBAF in THE (0.08 mL, 0.080 mmol) at 0° C. After stirring for 5 min at the same temperature, the mixture was concentrated in vacuo. The residue was purified by column chromatography (silica gel, CH₂Cl₂/MeOH=20/1) to obtain 3.11 (13 mg, 61% yield, over three steps) as a light-yellow solid.

¹H NMR (300 MHz, MeOD): δ 8.68 (1H, s, H-2), 8.04 (1H, s, H-8), 6.25 (1H, d, J=5.9 Hz, H-1′), 4.56 (1H, t, J=5.5 Hz, H-2′), 4.31 (1H, dd, J₁=5.2 Hz, J₂=3.5 Hz, H-3′), 4.11 (1H, q, J=3.2 Hz, H-4′), 3.86 (1H, dd, J₁=12.3 Hz, J₂=2.9 Hz, H-5′a), 3.76 (1H, dd, J₁=12.2 Hz, J₂=3.3 Hz, H-5′b), 3.69 (1H, s, CH), 3.33-3.25 (2H, m, CH₂), 1.36 (3H, t, J=7.6 Hz, CH₃). ¹³C NMR (75 MHz, MeOD): δ 167.1 (C-6), 152.6 (C-2), 151.3 (C-4), 133.5 (C-8), 118.5 (C-5), 97.6 (C-7), 90.1 (C-1′), 87.1 (C-4′), 81.6 (alkynyl-C), 77.3 (alkynyl-C), 76.0 (C-2′), 72.2 (C-3′), 63.1 (C-5′), 28.3 (CH₂), 14.3 (CH₃). HRMS (ESI⁺): m/z, [M+H]⁺ calcd for C₁₅H₁₈N₃O₄⁺, 304.1291; found, 304.1292.

6-Ethyl-7-vinyl-9-β-D-ribofuranosyl-7-deazapurine (3.1m). To a N2-purged solution of 3.10 (50 mg, 0.069 mmol) and Pd(PPh₃)₂C12 (2.4 mg, 0.003 mmol) in anhydrous DMF (2 mL) was added tributyl(vinyl)tin (22 μL, 0.083 mmol). The reaction mixture was stirred for 15 h at 100° C. The mixture was concentrated in vacuo. The residue was purified by column chromatography (silica gel, petroleum ether/EtOAc=5/1, 2/1) to afford protected 7-vinyl analogue (32 mg) which was dissolved in 7 M NH₃ in MeOH (5 mL) and stirred at room temperature overnight. The mixture was concentrated in vacuo. The residue was purified by column chromatography (silica gel, CH₂Cl₂/MeOH=20/1) to afford 3.1m (8 mg, 37% yield, over two steps) as a yellow solid.

¹H NMR (600 MHz, MeOD): δ 8.62 (1H, s, H-2), 7.90 (1H, d, J=0.8 Hz, H-8), 7.01 (1H, ddd, J₁=17.3 Hz, J₂=10.9 Hz, J₃=1.0 Hz, vinyl-CH), 6.25 (1H, d, J=6.1 Hz, H-1′), 5.67 (1H, dd, J₁=17.4 Hz, J₂=1.5 Hz, vinyl-CH₂a), 5.29 (1H, dd, J₁=10.9 Hz, J₂=1.6 Hz, vinyl-CH₂b), 4.59 (1H, dd, J₁=6.0 Hz, J₂=5.3 Hz, H-2′), 4.32 (1H, dd, J₁=5.3 Hz, J₂=3.4 Hz, H-3′), 4.11 (1H, q, J=3.2 Hz, H-4′), 3.87 (1H, dd, J₁=12.2 Hz, J₂=2.9 Hz, H-5′a), 3.77 (1H, dd, J₁=12.2 Hz, J₂=3.3 Hz, H-5′b), 3.13 (2H, q, J=7.6 Hz, CH₂), 1.36 (3H, t, J=7.6 Hz, CH₃). ¹³C NMR (151 MHz, MeOD): δ 166.2 (C-6), 152.1 (C-4), 151.5 (C-2), 129.4 (vinyl-C), 125.0 (C-8), 117.2 (C-5), 116.7 (C-7), 115.8 (viny-C), 90.0 (C-1′), 87.0 (C-4′), 75.8 (C-2′), 72.3 (C-3′), 63.2 (C-5′), 29.9 (CH₂), 14.1 (CH₃). HRMS (ESI⁺): m/z, [M+H]⁺ calcd for C₁₅H₂₀N₃O₄⁺, 306.1148; found, 306.1447.

6-Ethyl-7-cyclopropylethynyl-9-β-D-ribofuranosyl-7-deazapurine (3.1n). To a N2-purged solution of 3.10 (50 mg, 0.069 mmol), cyclopropylacetylene (0.06 mL, 0.697 mmol), Pd(PPh₃)₂C12 (2.4 mg) and CuI (1.3 mg) in anhydrous DMF (3 mL) was added Et₃N (0.03 mL, 0.209 mmol) and stirred for 15 h at 50° C. The mixture was concentrated in vacuo. The residue was purified by column chromatography (silica gel, petroleum ether/EtOAc=5/1, 2/1) to afford protected 7-cyclopropylethynyl analogue (33 mg) which was dissolved in 7 M NH₃ in MeOH (5 mL) and stirred at room temperature overnight. The mixture was concentrated in vacuo. The residue was purified by column chromatography (silica gel, CH₂Cl₂/MeOH=20/1) to afford 3.1n (12 mg, 50% yield, over two steps) as a yellow solid. ¹H NMR (600 MHz, MeOD): δ 8.63 (1H, s, H-2), 7.83 (1H, s, H-8), 6.21 (1H, d, J=5.9 Hz, H-1′), 4.53 (1H, t, J=5.6 Hz, H-2′), 4.28 (1H, dd, J₁=5.2 Hz, J₂=3.4 Hz, H-3′), 4.09 (1H, q, J=3.2 Hz, H-4′), 3.84 (1H, dd, J₁=12.2 Hz, J₂=3.0 Hz, H-5′a), 3.74 (1H, dd, J₁=12.2 Hz, J₂=3.3 Hz, H-5′b), 3.25 (2H, q, J=7.6 Hz, CH₂), 1.54-1.48 (1H, m, CH), 1.34 (3H, t, J=7.6 Hz, CH₃), 0.92-0.88 (2H, m, CH₂), 0.76-0.73 (2H, m, CH₂). ¹³C NMR (151 MHz, MeOD): δ 167.0 (C-6), 152.3 (C-2), 151.2 (C-4), 131.8 (C-8), 118.7 (C-5), 99.2 (C-7), 96.6 (alkynyl-C), 90.0 (C-1′), 87.0 (C-4′), 75.9 (C-2′), 72.3 (C-3′), 68.7 (alkynyl-C), 63.1 (C-5′), 28.4 (CH₂), 14.5 (CH₃), 8.9 ((CH₂)₂), 0.9 (CH). HRMS (ESI⁺): m/z, [M+H]⁺ calcd for C₁₈H₂₂N₃O₄⁺, 344.1604; found, 344.1610.

6-Ethyl-7-iodo-9-β-D-ribofuranosyl-7-deazapurine (3.10). Compound 3.10 (320 mg, 0.446 mmol) was dissolved in 7 M NH₃ in MeOH (10 mL) and stirred at room temperature overnight. The mixture was concentrated in vacuo. The residue was purified by column chromatography (silica gel, CH₂Cl₂/MeOH=20/1) to afford 3.10 (134 mg, 74% yield) as a yellow solid.

¹H NMR (300 MHz, MeOD): δ 8.67 (1H, s, H-2), 7.98 (1H, s, H-8), 6.27 (1H, d, J=5.9 Hz, H-1′), 4.53 (1H, t, J=5.6 Hz, H-2′), 4.29 (1H, dd, J₁=5.3 Hz, J₂=3.4 Hz, H-3′), 4.10 (1H, q, J=3.2 Hz, H-4′), 3.85 (1H, dd, J₁=12.2 Hz, J₂=3.0 Hz, H-5′a), 3.75 (1H, dd, J₁=12.3 Hz, J₂=3.2 Hz, H-5′b), 3.37-3.29 (2H, overlapped with MeOD, CH₂), 1.36 (3H, t, J=7.6 Hz, CH₃). ¹³C NMR (75 MHz, MeOD): δ 166.5 (C-6), 151.8 (C-2), 151.8 (C-4), 134.1 (C-8), 119.2 (C-5), 89.7 (C-1′), 87.0 (C-4′), 76.0 (C-2′), 72.2 (C-3′), 63.0 (C-5′), 52.7 (C-7), 27.2 (CH₂), 14.9 (CH₃). HRMS (ESI⁺): m/z, [M+H]⁺ calcd for C₁₃H₁₇I₁N₃O₄⁺, 406.0260; found, 406.0253.

6-Ethyl-7-chloro-9-β-D-ribofuranosyl-7-deazapurine (3.1p). To a mixture of 3.9 (130 mg, 0.219 mmol) in anhydrous DMF (4 mL) was added dropwise NCS (35 mg, 0.263 mmol) in anhydrous DMF (1 mL) and stirred at room temperature overnight. To the mixture was added water and EtOAc and the aqueous layer was extracted with EtOAc. The combined organic layer was washed with water, brine, dried over MgSO₄, filtered and concentrated in vacuo. The residue was purified by column chromatography (silica gel, petroleum ether/EtOAc=3/1, 2/1) to afford protected 6-ethyl-7-chloro analogue (50 mg) which was dissolved in 7 M NH₃ in MeOH (5 mL) and stirred overnight at room temperature. The mixture was concentrated in vacuo. The residue was purified by column chromatography (silica gel, CH₂Cl₂/MeOH=20/1) to afford 3.1p (25 mg, 36% yield, over two steps) as a white solid.

¹H NMR (600 MHz, MeOD): δ 8.67 (1H, s, H-2), 7.85 (1H, s, H-8), 6.29 (1H, d, J=5.8 Hz, H-1′), 4.52 (1H, t, J=5.5 Hz, H-2′), 4.29 (1H, dd, J₁=5.2 Hz, J₂=3.5 Hz, H-3′), 4.10 (1H, q, J=3.2 Hz, H-4′), 3.84 (1H, dd, J₁=12.2 Hz, J₂=3.0 Hz, H-5′a), 3.76 (1H, dd, J₁=12.2 Hz, J₂=3.3 Hz, H-5′b), 3.26 (2H, qd, J₁=7.7 Hz, J2=0.9 Hz, CH₂), 1.37 (3H, t, J=7.6 Hz, CH₃). 13C NMR (151 MHz, MeOD): δ 166.2 (C-6), 152.5 (C-2), 150.9 (C-4), 125.7 (C-8), 115.7 (C-5), 105.8 (C-7), 89.6 (C-1′), 87.0 (C-4′), 76.1 (C-2′), 72.2 (C-3′), 63.0 (C-5′), 28.5 (CH₂), 14.4 (CH₃). HRMS (ESI⁺): m/z, [M+H]⁺ calcd for C₁₃H₁₇Cl₁N₃O₄⁺, 314.0902; found, 314.0901.

6-Ethyl-7-bromo-9-β-D-ribofuranosyl-7-deazapurine (3.1q). To a mixture of 3.9 (130 mg, 0.219 mmol) in anhydrous DMF (4 mL) was added dropwise NBS (47 mg, 0.263 mmol) in anhydrous DMF (1 mL) and stirred at the room temperature overnight. To the mixture was added water and EtOAc and the aqueous layer was extracted with EtOAc. The combined organic layer was washed with water, brine, dried over MgSO₄, filtered and concentrated in vacuo. The residue was purified by column chromatography (silica gel, petroleum ether/EtOAc=3/1, 2/1) to afford protected 6-ethyl-7-bromo analogue (91 mg) which was dissolved in 7 M NH₃ in MeOH (5 mL) and stirred overnight at room temperature. The mixture was concentrated in vacuo. The residue was purified by column chromatography (silica gel, CH₂Cl₂/MeOH=20/1) to afford 3.1q (35 mg, 44% yield, over two steps) as a white solid.

¹H NMR (600 MHz, MeOD): δ 8.66 (1H, s, H-2), 7.90 (1H, s, H-8), 6.28 (1H, d, J=5.8 Hz, H-1′), 4.53 (1H, t, J=5.6 Hz, H-2′), 4.30 (1H, dd, J₁=5.3 Hz, J₂=3.5 Hz, H-3′), 4.10 (1H, q, J=3.2 Hz, H-4′), 3.85 (1H, dd, J₁=12.2 Hz, J₂=3.0 Hz, H-5′a), 3.76 (1H, dd, J₁=12.2 Hz, J₂=3.3 Hz, H-5′b), 3.27 (2H, qd, J₁=7.6 Hz, J2=1.4 Hz, CH₂), 1.35 (3H, t, J=7.6 Hz, CH₃). 13C NMR (151 MHz, MeOD): δ 166.3 (C-6), 152.2 (C-2), 151.2 (C-4), 128.3 (C-8), 116.9 (C-5), 89.7 (C-1′), 89.5 (C-7), 87.0 (C-4′), 76.1 (C-2′), 72.2 (C-3′), 63.0 (C-5′), 28.0 (CH₂), 14.5 (CH₃). HRMS (ESI⁺): m/z, [M+H]⁺ calcd for C₁₃H₁₇Br₁N₃O₄⁺, 358.0397; found, 358.0397.

6-Ethyl-7-Fluoro-9-β-D-ribofuranosyl-7-deazapurine (3.1r). To a solution of 3.12¹⁹ (100 mg, 0.162 mmol), Fe(acac)3 (5.7 mg, 0.016 mmol), CuI (6.2 mg, 0.032 mmol) and NMP (0.50 mL) in anhydrous THE (5 mL) was added dropwise 3M ethylMgBr in THE (1.12 mL) at 0° C. and the reaction mixture was stirred for 45 min at room temperature. The reaction was quenched with sat. aqueous NH₄Cl and extracted with EtOAc. The organic layer was dried over MgSO₄, filtered and concentrated in vacuo. The residue was purified by column chromatography (silica gel, petroleum ether/EtOAc=5/1, 3/1) to afford crude protected 6-ethyl-7-fluoro analogue (quantitative). This compound was dissolved in 7 M NH₃ in MeOH (5 mL) and the mixture was stirred overnight at room temperature. The mixture was concentrated in vacuo. The residue was purified by column chromatography (silica gel, CH₂Cl₂/MeOH=20/1) to afford 3.1r (20 mg, 41% yield, over two steps) as a white solid.

¹H NMR (300 MHz, MeOD): δ 8.67 (1H, s, H-2), 7.63 (1H, d, J=2.1 Hz, H-8), 6.32 (1H, d, J=5.9 Hz, H-1′), 4.49 (1H, t, J=5.6 Hz, H-2′), 4.28 (1H, dd, J₁=5.3 Hz, J2=3.3 Hz, H-3′), 4.08 (1H, q, J=3.2 Hz, H-4′), 3.83 (1H, dd, J₁=12.2 Hz, J₂=3.1 Hz, H-5′a), 3.74 (1H, dd, J₁=12.1 Hz, J₂=3.4 Hz, H-5′b), 3.09 (2H, q, J=7.6 Hz, CH₂), 1.37 (3H, t, J=7.6 Hz, CH₃). ¹³C NMR (151 MHz, MeOD): δ 165.0, 165.0 (C-6), 152.7 (C-2), 147.7 (C-4), 145.0, 143.4 (C-7), 110.8, 110.6 (C-8), 108.6, 108.5 (C-5), 89.3 (C-1′), 86.9 (C-4′), 76.0 (C-2′), 72.2 (C-3′), 63.1 (C-5′), 29.6 (CH₂), 13.7 (CH₃). HRMS (ESI⁺): m/z, [M+H]⁺ calcd for C₁₃H₁₇F₁N₃O₄⁺, 298.1197; found, 298.1201.

### Human Norovirus Antiviral Assay

The cytotoxicity was investigated by the MTS method, by exposing uninfected cells to the same concentrations of compounds for 3 days. The % cell viability was calculated as (OD_{treated}/OD_{cc})×100, where OD_{cc} is the OD of uninfected untreated cells and OD treated are uninfected cells treated with compounds. The CC₅₀ was defined as the compound concentration that reduces the number of viable cells by 50%.

HG23 cells (5000/well) were seeded into the wells of 96-well plates in complete DMEM without G418. After 24 h of incubation, serial dilutions of compounds were added. Cells were further incubated for 72 h, after which cell culture supernatant was removed and monolayers were washed with phosphate-buffered saline (PBS). Cell monolayers were collected for RNA load quantification by quantitative reverse transcription PCR (qRT PCR). To determine relative levels of Norwalk virus replicon RNA, β-actin was used as a normalizer and ratios were calculated by the Pfaffl method.

The Norwalk virus replicon/β-actin ratio was calculated as follows: Ratio=E_{Norwalk}^{ΔCT, Norwalk (CC-TC)}/E_{β-actin}^{ΔCT, β-actin (CC-TC), where E}_{Norwalk} and E_{β-actin} represent the amplification efficiencies (E=10^{-1/slope}) of the Norwalk virus replicon and β-actin qRT PCRs, respectively; ΔC_{T}, Norwalk virus (CC-CT) is the C_{T} (threshold cycle) of untreated control cells (CC) minus the ΔC_{T}, β-actin (CC-CT) is the C_{T} of untreated control cells (CC) minus the Cr of cells treated with a compound concentration (TC) obtained with β-actin primers and probe. Efficiency values (E_{Norwalk} and E_{β-actin}) were determined for each qRT PCR. The 50% effective concentrations were defined as the compound concentrations that results in 50% reductions of the relative Norwalk virus replicon RNA levels.

### Example 5. Anti-Influenza Virus Activity

The antiviral activity of 3.1r against other subtypes of influenza A (H3N2 and H5N1) and influenza B viruses was further investigated (Table 4), revealing 3.1r as a potent compound with activity in the submicromolar range. Although cytotoxicity was observed to some extent, the high selectivity provided strong evidence for 3.1r as a promising candidate against influenza.

TABLE 4
Antiviral activity of derivative 3.1r against
influenza A (H1N1) virus in MDCK cells.

| Compd | Virus | Cell line | EC₅₀(μM)^{a} | CC₅₀(μM)^{b} | SI₅₀^{c} |
|---|---|---|---|---|---|
| | Influenza A (H1N1) | MDCK | 0.34 | 43 | >130 |
| | Influenza A (H3N2) | MDCK | <0.1 | 30 | >300 |
| 3.1r | Influenza A (H5N1) | MDCK | <0.1 | 18 | >180 |
| | Influenza B | MDCK(Brisbane | <0.1 | 25 | >250 |
| | Influenza B | MDCK(Florida) | <0.1 | 77 | >770 |
| Ribavirin | Influenza A (H1N1) | MDCK | 10.2 | >4095 | >400 |

^{a}Effective concentration that reduces viral replication by 50%;
^{b}Cytotoxic concentration that reduces cell viability by 50%;
^{c}SI₅₀: CC₅₀/EC₅₀.

### Example 6. Antiviral Activity Against Other RNA Viruses

In a subsequent study, the antiviral activity and cytotoxicity of reference compound 3.1a and compounds of the invention 3.1b-q against chikungunya virus (S27 strain, in Vero 76 cells), dengue virus 2 (New Guinea C, in Huh7 cells), yellow fever virus (YFV 17D strain, in Huh7 cells), and enterovirus-71 (Taiwan/4643/98, in Vero 76 cells) were also investigated. Infergen (for chikungunya virus, DENY, and YFV assays), M128533 (for MERS-CoV assay), 2′-fluoro-2′-deoxycytidine (for measles virus assay), ribavirin (for tacaribe virus assay), and pirodavir (for enterovirus-71 assay) were used as positive controls in cytopathic effect (CPE) assays.

Screening against chikungunya virus indicated that 7-fluoro-6-ethyl analogue 3.1q possessed good activity and selectivity with an EC₅₀ value of 0.87 μM and SI₅₀ of 70, respectively (see results in Table 5). Unfortunately, the other compounds (3.1b-p) exhibited no activity against chikungunya virus; reference compound 3.1a was also inactive. Interestingly, compound 3.2b was also inactive. These results indicated that the introduction of a fluorine atom at the 7-position may be beneficial to increase antiviral activity against RNA viruses. The 7-fluoro derivative 3.1r also displayed low micromolar to submicromolar activity against dengue virus 2 and MERS-CoV. However, other 6,7-disubstitued 7-deazaadenosines (3.11-q) showed no significant activity.

The 6-cyclopropylethynyl analogue 3.1c was active against dengue virus 2, yellow fever virus, MERS-CoV, measles virus, and tacaribe virus with EC₅₀ values ranging from 0.43 to 10 μM. In particular, compound 3.1c displayed significant activity with good selectivity against measles virus and tacaribe virus (EC₅₀=0.97 and 0.43 μM, SI₅₀s>100 and >230, respectively). The 6-cyclopentylethynyl congener 3.1g displayed low micromolar activity (EC₅₀=0.32 μM) against tacaribe virus, however its cytotoxicity (CC₅₀=34 μM) was slightly higher than that of 3.1c (CC₅₀>100 μM). Unfortunately, the other 6-substituted derivatives were inactive against tacaribe virus (Table 1). On the other hand, derivatives 3.1e-q showed micromolar activity with low selectivity against measles virus (Table 1). Likewise, a submicromolar to micromolar activity was observed for compound 3.1g against dengue virus 2, YFV, MersCoV, measles virus and tacaribe virus, with 3.1g being more cytotoxic than 3.1c. Among 6-substituted derivatives (3.1b-j), the introduction of cyclic alkyl substituted ethynyl groups resulted in compounds with good activity and selectivity.

Derivatives 3.1h-j bearing substituted phenyl groups were active against YFV at low micromolar concentrations (Table 1). Compounds 3.1f and 3.1g exhibited low micromolar activity and moderate selectivity against enterovirus-71 (EC₅₀=2.2 and 1.8 μM, SI₅₀>45 and 36, respectively), while 3.1c was inactive (Table 7). Their activity was further confirmed in a secondary assay (Table 8). Based on the good activity in the preliminary screening, the activity of compound 3.1c against tacaribe virus was further demonstrated in a secondary assay (Table 9); an EC₅₀ of 1.1 μM was observed for compound 3.1c, which outperformed ribavirin. Antiviral assay against other RNA viruses General procedure of reduction of virus-Induced cytopathic effect (primary CPE assay). The antiviral screening was performed at the Institute of Antiviral Research, Utah State University according to a procedure adapted and slightly modified from a previous report.²⁰ Confluent or near-confluent cell culture monolayers of Vero 76 cells (or other appropriate cell line) are prepared in 96-well disposable microplates the day before testing. Cells are maintained in MEM supplemented with 5% FBS. For antiviral assays the same medium is used but with FBS reduced to 2% and supplemented with 50-μg/ml gentamicin. Compounds are dissolved in DMSO, saline or the diluent requested by the submitter. Less soluble compounds may be vortexed, heated, and/or sonicated, and if they still do not go into solution are tested as colloidal suspensions. The test compound is prepared at four serial log 10 concentrations, usually 100, 10, 1.0, and 0.1 μg/ml or μM (or per sponsor preference). Lower concentrations are used when insufficient compound is supplied or when a lower starting concentration is requested. Five microwells are used per dilution: three for infected cultures and two for uninfected toxicity cultures. Controls for the experiment consist of six microwells that are infected and not treated (virus controls) and six that are untreated and uninfected (cell controls) on every plate. A known active drug is tested in parallel as a positive control drug using the same method as is applied for test compounds. The positive control is tested with every test run.

On the testing day, the growth media is removed from the cells and the test compound is applied in 0.1 ml volume to wells at 2× concentration. Virus, normally at a titre that will cause >80% CPE (usually an MOI <0.003), in 0.1 ml volume is added to the wells designated for virus infection. Medium devoid of virus is placed in toxicity control wells and cell control wells. Plates are incubated at 37° C. with 5% CO₂ until marked CPE (>80% CPE for most virus strains) is observed in virus control wells. The plates are then stained with 0.011% neutral red for approximately two hours at 37° C. in a 5% CO₂ incubator. The neutral red medium is removed by complete aspiration, and the cells may be rinsed 1× with phosphate buffered solution (PBS) to remove residual dye. The PBS is completely removed, and the incorporated neutral red is eluted with 50% Sorensen's citrate buffer/50% ethanol for at least 30 minutes. Neutral red dye penetrates into living cells, thus, the more intense the red color, the larger the number of viable cells present in the wells. The dye content in each well is quantified using a spectrophotometer at 540 nm wavelength. The dye content in each set of wells is converted to a percentage of dye present in untreated control wells using a Microsoft Excel-based spreadsheet and normalized based on the virus control. The 50% effective (EC₅₀, virus-inhibitory) concentrations and 50% cytotoxic (CC₅₀, cell-inhibitory) concentrations are then calculated by regression analysis. The quotient of CC₅₀ divided by EC₅₀ gives the selectivity index (SI) value. Compounds showing SI values >10 are considered active.

The VYR test is a direct determination of how much the test compound inhibits virus replication. Virus yielded in the presence of test compound is titrated and compared to virus titres from the untreated virus controls. Titration of the viral samples (collected as described in the paragraph above) is performed by endpoint dilution.²¹ Serial 10-fold dilutions of supernatant are made and plated into 4 replicate wells containing fresh cell monolayers of Vero 76 cells. Plates are then incubated, and cells are scored for presence or absence of virus after distinct CPE is observed, and the CCID₅₀ calculated using the Reed-Muench method²¹. The 90% (one log₁₀) effective concentration (EC₉₀) is calculated by regression analysis by plotting the logo of the inhibitor concentration versus login of virus produced at each concentration. Dividing EC₉₀ by the CC₅₀ gives the SI value for this test.

General procedure of reduction of virus yield (Secondary VYR assay). Active compounds may be further tested in a confirmatory VYR assay. This assay is set up similar to the methodology described above, except that eight half-log 10 concentrations of compound are tested for antiviral activity and cytotoxicity. After sufficient virus replication occurs (generally 3 days for many viruses), a sample of supernatant is taken from each infected well (replicate wells are pooled) and tested immediately or held frozen at −80° C. for later virus titre determination. After maximum CPE is observed, the viable plates are stained with neutral red dye. The incorporated dye content is quantified as described above to generate the EC₅₀ and CC₅₀ values.

Procedure details for in vitro screening with several viruses, including virus strain, cell line, control, Incubation days: influenza A virus (California/07/2009; in MDCK cells; positive control, Ribavirin; Incub. Days, 6), respiratory syncytial virus (A2; in RD cells; positive control, Ribavirin; Incub. days, 6), chikungunya virus (S27 strain; in Vero 76 cells; positive control, Infergen; Incub. Days, 4), dengue virus 2 (New Guinea C; in Huh7 cells; positive control, Infergen; Incub. Days, 6), yellow fever virus (YFV 17D strain; in Huh7 cells; positive control, Infergen; Incub. Days, 5), MERS-CoV (EMC strain; in Vero 76 cells; positive control, M-128533; Incub. Days, 5), measles virus (CC strain; in Vero 76 cells; positive control, 2′-Fluoro-2′-deoxycytidine; Incub. Days, 6), tacaribe virus (TRVL11573 strain; in Vero cells; positive control, Ribavirin; Incub. Days, 7) and enterovirus-71 (Taiwan/4643/98; in Vero 76 cells; positive control, Pirodavir; Incub. Days, 2).

TABLE 5
Activity and cytotoxicity of reference compound 3.1a
and derivatives according to the invention against chikungunya
virus (S₂₇ strain) in Vero 76 cells.

| Compd | Assay name | EC₅₀ (μM)^{a} | CC₅₀ (μM)^{b} | SI₅₀^{c} |
|---|---|---|---|---|
| | Visual | >100 | >100 | 0 |
| 3.1a | Neutral Red | >100 | >100 | 0 |
| | Visual | >100 | >100 | 0 |
| 3.1b | Neutral Red | >37 | 37 | 0 |
| | Visual | >100 | >100 | 0 |
| 3.1c | Neutral Red | >100 | >100 | 0 |
| | Visual | >100 | >100 | 0 |
| 3.1d | Neutral Red | >8.8 | 8.8 | 0 |
| | Visual | >100 | >100 | 0 |
| 3.1e | Neutral Red | >100 | >100 | 0 |
| | Visual | 41 | >100 | >2 |
| 3.1f | Neutral Red | >100 | >100 | 0 |
| | Visual | >100 | >100 | 0 |
| 3.1g | Neutral Red | >100 | >100 | 0 |
| | Visual | >36 | 36 | 0 |
| 3.1h | Neutral Red | 32 | 46 | 1 |
| | Visual | >32 | 32 | 0 |
| 3.1i | Neutral Red | >30 | 30 | 0 |
| | Visual | >32 | 32 | 0 |
| 3.1j | Neutral Red | >23 | 23 | 0 |
| | Visual | >100 | >100 | 0 |
| 3.1l | Neutral Red | >100 | >100 | 0 |
| | Visual | >100 | >100 | 0 |
| 3.1m | Neutral Red | >100 | >100 | 0 |
| | Visual | >100 | >100 | 0 |
| 3.1n | Neutral Red | >100 | >100 | 0 |
| | Visual | >100 | >100 | 0 |
| 3.1o | Neutral Red | >100 | >100 | 0 |
| | Visual | >100 | >100 | 0 |
| 3.1p | Neutral Red | >100 | >100 | 0 |
| | Visual | >100 | >100 | 0 |
| 3.1q | Neutral Red | >100 | >100 | 0 |
| | Visual | 0.000042^{d} | >0.01^{d} | >240 |
| Infergen | Neutral Red | 0.000037^{d} | >0.01^{d} | >270 |

^{a}Effective concentration that reduces viral replication by 50%;
^{b}Cytotoxic concentration that reduces cell viability by 50%;
^{c}SI₅₀: CC₅₀/EC₅₀;
^{d}Concentration unit: μg/mL.

TABLE 6
Activity and cytotoxicity of reference compound 3.1a
and derivatives according to the invention against
dengue virus 2 (New Guinea C strain) in Huh7 cells.

| Compd | Assay name | EC₅₀ (μM)^{a} | CC₅₀ (μM)^{b} | SI₅₀^{c} |
|---|---|---|---|---|
| | Visual | 32 | >100 | >3 |
| 3.1a | Neutral Red | 45 | >100 | >2 |
| | Visual | 2.7 | 8.6 | 3 |
| 3.1b | Neutral Red | 1.3 | 8.5 | 6 |
| | Visual | 0.32 | 3.6 | 11 |
| 3.1d | Neutral Red | 0.37 | 3.7 | 10 |
| | Visual | 3.2 | 24 | 7 |
| 3.1e | Neutral Red | 1.8 | 21 | 12 |
| | Visual | 0.75 | 12 | 16 |
| 3.1f | Neutral Red | 0.96 | 11 | 11 |
| | Visual | >32 | 32 | 0 |
| 3.1h | Neutral Red | >29 | 29 | 0 |
| | Visual | 2.6 | 24 | 9 |
| 3.1i | Neutral Red | 2.7 | 22 | 8 |
| | Visual | 1.1 | 12 | 11 |
| 3.1j | Neutral Red | 1 | 13 | 13 |
| | Visual | >100 | >100 | 0 |
| 3.1l | Neutral Red | >51 | 51 | 0 |
| | Visual | 32 | 42 | 1 |
| 3.1m | Neutral Red | >19 | 19 | 0 |
| | Visual | 13 | 52 | 4 |
| 3.1n | Neutral Red | >8.3 | 8.3 | 0 |
| | Visual | >68 | 68 | 0 |
| 3.1o | Neutral Red | >64 | 64 | 0 |
| | Visual | 32 | 68 | 2 |
| 3.1p | Neutral Red | >38 | 38 | 0 |
| | Visual | >68 | 68 | 0 |
| 3.1q | Neutral Red | >47 | 47 | 0 |
| | Visual | <0.00001^{d} | >0.01^{d} | >1000 |
| Infergen | Neutral Red | 0.000019^{d} | >0.01^{d} | >530 |

^{a}Effective concentration that reduces viral replication by 50%;
^{b}Cytotoxic concentration that reduces cell viability by 50%;
^{c}SI₅₀: CC₅₀/EC₅₀;
^{d}Concentration unit: μg/mL.

TABLE 7
Activity and cytotoxicity of reference compound 3.1a
and derivatives according to the invention against enterovirus-71
(Tainan/4643/98 strain) in Vero 76 cells.

| Compd | Assay name | EC₅₀ (μM)^{a} | CC₅₀ (μM)^{b} | SI₅₀^{c} |
|---|---|---|---|---|
| | Visual | >100 | >100 | 0 |
| 3.1a | Neutral Red | >100 | >100 | 0 |
| 3.1b | Visual | 90 | >100 | >1 |
| | Neutral Red | >84 | 84 | 0 |
| | Visual | >100 | >100 | 0 |
| 3.1c | Neutral Red | >100 | >100 | 0 |
| | Visual | >100 | >100 | 0 |
| 3.1d | Neutral Red | >8 | 8 | 0 |
| | Visual | 4.2 | >100 | >24 |
| 3.1e | Neutral Red | 4.3 | >100 | >23 |
| | Visual | 1.6 | >100 | >63 |
| 3.1f | Neutral Red | 2.2 | >100 | >45 |
| | Visual | 1.5 | >100 | >63 |
| 3.1g | Neutral Red | 1.8 | 64 | 36 |
| | Visual | >39 | 39 | 0 |
| 3.1h | Neutral Red | >46 | 46 | 0 |
| | Visual | >32 | 32 | 0 |
| 3.1i | Neutral Red | >28 | 28 | 0 |
| | Visual | >32 | 32 | 0 |
| 3.1j | Neutral Red | >31 | 31 | 0 |
| | Visual | >100 | >100 | 0 |
| 3.1l | Neutral Red | >100 | >100 | 0 |
| | Visual | >100 | >100 | 0 |
| 3.1m | Neutral Red | >100 | >100 | 0 |
| | Visual | >100 | >100 | 0 |
| 3.1n | Neutral Red | >100 | >100 | 0 |
| | Visual | >100 | >100 | 0 |
| 3.1o | Neutral Red | >100 | >100 | 0 |
| | Visual | >100 | >100 | 0 |
| 3.1p | Neutral Red | >100 | >100 | 0 |
| | Visual | >100 | >100 | 0 |
| 3.1q | Neutral Red | >100 | >100 | 0 |
| | Visual | >100 | >100 | 0 |
| 3.1r | Neutral Red | >100 | >100 | 0 |
| | Visual | 0.40 | >27 | >67 |
| Pirodavir | Neutral Red | 0.27 | >27 | >100 |

^{a}EC. reducing viral replication by 50%;
^{b}CC: Reducing cell viability by 50%;
^{c}SI₅₀: CC₅₀/EC₅₀.

TABLE 8
Activity and cytotoxicity of derivatives of the invention
3.1e-g against enterovirus-71 (Tainan/4643/98 strain)
in Vero 76 cells. (Secondary assay)

| | | EC₅₀ | EC₉₀ | CC₅₀ | |
|---|---|---|---|---|---|
| Compd | Assay name | (μM)^{a} | (μM)^{b} | (μM)^{c} | SI₅₀^{d} |
| 3.1e | Visual (Virus yield reduction)/ | — | 4.6 | 96 | — |
| | Neutral Red (Toxicity) | | | | |
| | Neutral Red (Cytopathic | 4 | — | 96 | 24 |
| | effect/Toxicity) | | | | |
| 3.1f | Visual (Virus yield reduction)/ | — | 17 | 80 | — |
| | Neutral Red (Toxicity) | | | | |
| | Neutral Red (Cytopathic | 29 | — | 80 | 3 |
| | effect/Toxicity) | | | | |
| 3.1g | Visual (Virus yield reduction)/ | — | 1.1 | 39 | — |
| | Neutral Red (Toxicity) | | | | |
| | Neutral Red (Cytopathic | 3.8 | — | 39 | 10 |
| | effect/Toxicity) | | | | |
| Pirodavir | Visual (Virus yield reduction)/ | — | 0.46 | >27 | — |
| | Neutral Red (Toxicity) | | | | |
| | Neutral Red (Cytopathic | 0.35 | — | >27 | >77 |
| | effect/Toxicity) | | | | |

^{a}Effective concentration that reduces viral replication by 50%;
^{b}Effective concentration that reduces viral replication by 90%;
^{c}Cytotoxic concentration that reduces cell viability by 50%;
^{d}SI₅₀: CC₅₀/EC₅₀.

TABLE 9
Activity and cytotoxicity of derivative according to the invention 3.1c against
tacaribe virus (TRVL11573 strain) in Vero cells. (Secondary assay)

| | | EC₅₀ | EC₉₀ | CC₅₀ | |
|---|---|---|---|---|---|
| Compd | Assay name | (μM)^{a} | (μM)^{b} | (μM)^{c} | SI₅₀^{d} |
| 3.1c | Visual (Virus yield reduction)/ | / | 0.65 | 100 | / |
| | Neutral Red (Toxicity) | | | | |
| | Neutral Red (Cytopathic | 1.1 | / | 95 | 86 |
| | effect/Toxicity) | | | | |
| Ribavirin | Visual (Virus yield reduction)/ | / | 11.5 | >2948 | / |
| | Neutral Red (Toxicity) | | | | |
| | Neutral Red (Cytopathic | 28.7 | / | >3603 | >130 |
| | effect/Toxicity) | | | | |

^{a}Effective concentration that reduces viral replication by 50%;
^{b}Effective concentration that reduces viral replication by 90%;
^{c}Cytotoxic concentration that reduces cell viability by 50%;
^{d}SI₅₀: CC₅₀/EC₅₀

### REFERENCES

1. Anzai et al. J. Antibiot. 1957, 10, 201-204.
2. Acs et al. Proc. Nat. Acad. Sci. U.S.A 1964, 52, 493-501.
3. Olsen et al. Antimicrob. Agents. Chemother. 2004, 48 (10), 3944-3953.
4. Naǔs et al. J. Med. Chem. 2010, 53 (1), 460-470.
5. Li et al. J. Org. Chem. 2020, 85 (2), 403-418.
6. Wu et al. J. Med. Chem. 2010, 53 (22), 7958-7966.
7. Bourderioux et al. J. Med. Chem. 2011, 54 (15), 5498-5507.
8. Naǔs et al. J. Med. Chem. 2014, 57 (3), 1097-1110.
9. Eldrup et al. J. Med. Chem. 2004, 47, 5284-5297.
10. Naǔs et al. Bioorg. Med. Chem. 2012, 20 (17), 5202-5214.
11. Perlíková et al. Chem med chem 2013, 8 (5), 832-846.
12. Perlíková & Hocek Med. Res. Rev. 2017, 37 (6), 1429-1460.
13. Milisavljevic et al. ACS Infect. Dis. 2021, 7 (2), 471-478.
14. Seela & Ming Tetrahedron 2007, 63 (39), 9850-9861.
15. Seela & Peng J. Org. Chem. 2006, 71 (1), 81-90.
16. Ahmed et al. Nucleosides Nucleotides Nucleic Adds 2019, 38 (8), 590-603.
17. Sonogashira & Hagihara Tetrahedron Lett. 1975, 55, 4467-4470.
18. Chinchilla & Nájera Chem. Soc. Rev. 2011, 40 (10), 5084-5121.
19. Wang et al. Nucleosides Nucleotides and Nudeic Adds 2004, 23 (1-2), 161-170.
20. Puhl, A. C et al. ACS omega 2021, 6(11), 7454-7468.
21. Reed, L. J.; Muench, H. Am. J. Hyg. 1938, 27, 493-497.

What is claimed is:

1. A compound with general structure (I) or (II), or a pharmaceutically acceptable salt or prodrug thereof: 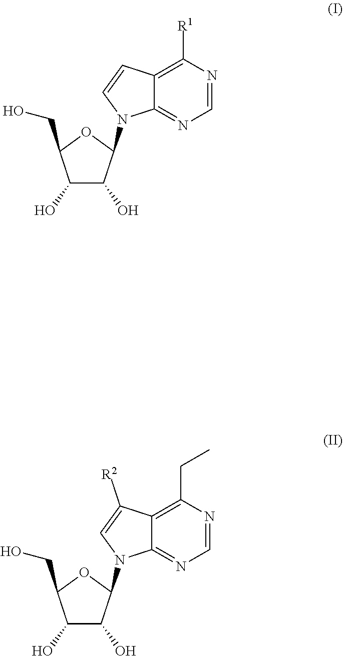
wherein R¹ is selected from:
i. vinyl, vinylC₁₋₆alkyl or C₁₋₆alkylene-vinyl, wherein the vinyl, vinylC₁₋₆alkyl or C₁₋₆alkylene-vinyl is optionally substituted with OC₁₋₆alkyl, OH, halogen, amino, dimethylamino or C₆₋₁₀aryl,
ii. ethynyl or an ethynyl alkyl, wherein the alkyl group is a straight or branched C1 to C6 chain,
iii. ethynyl cycloalkyl, wherein the cycloalkyl is a C3-C7 cycloalkyl, and
iv. ethynyl benzene, wherein the benzene ring is optionally substituted with an halogen;
R² is selected from: a C2-C6 chain comprising a vinyl or ethynyl group, the C2-C6 chain optionally substituted with a C3-C6 cycloalkyl, or the C2-C6 chain optionally substituted with a benzene ring wherein the benzene ring is optionally substituted with a halogen, OH or CHs, and a halogen.

2. The compound or a pharmaceutically acceptable salt or prodrug thereof according to claim 1, wherein R¹ is selected from: vinyl, vinylC₁₋₄alkyl, C₁₋₄alkylene-vinyl, ethynylC₁₋₄alkyl, ethynylC₃₋₅cycloalkyl, and ethynyl benzene.

3. The compound or a pharmaceutically acceptable salt or prodrug thereof according to claim 1, wherein R¹ is selected from:
i. vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl, wherein said vinyl, prop-1-enyl, 3-methylbut-1-enyl, but-1-enyl, pent-1-enyl, hex-1-enyl, 4-methylpent-1-enyl, hept-1-enyl, 5-methylhex-1-enyl, 4-methylhex-1-enyl, 3-methylhex-1-enyl, oct-1-enyl, 3,3-dimethylbut-1-enyl, 6-methylhept-1-enyl, 5-methylhept-1-enyl, 4-methylhept-1-enyl, 3-methylhept-1-enyl, 2-methylhept-1-enyl is optionally substituted with OC₁₋₄alkyl, OH, halogen, or C₆₋₁₀aryl,
ii. ethynyl, prop-1-ynyl, buty-1-ynyl, pent-1-ynyl, 3-methylbut-1-ynyl, hex-1-ynyl, 4-methylpent-1-ynyl, 3-methylpent-1-ynyl, hept-1-ynyl, 5-methyl hex-1-ynyl, 4-methylhex-1-ynyl, 3-methylhex-1-ynyl, 4,4-dimethylpent-1-ynyl, oct-1-ynyl, 6-methylhept-1-ynyl, 5-methylhept-1 ynyl, 4-methylhept-1-ynyl, 3-methylhept-1-ynyl, 2-cyclopropylethynyl, 2-cyclobutylethynyl, 2-cyclopentylethynyl, 2-cyclohexylethynyl, 2-cycloheptylethynyl, and
iii. ethynyl benzene, wherein the benzene ring is optionally substituted with an halogen.

4. The compound or a pharmaceutically acceptable salt or prodrug thereof according to claim 1, wherein R² is selected from: an optionally substituted C2-C4 chain comprising a vinyl or ethynyl group, F, Cl, Br and I.

5. The compound or a pharmaceutically acceptable salt or prodrug thereof according to claim 1, wherein R² is selected from: vinyl, vinylC₁₋₄alkyl, C₁₋₄allylene-vinyl, ethynyl, ethynylC₁₋₄alkyl, C₁₋₄alkylene-ethynyl, F, Cl, Br and I; wherein said vinyl, vinylC₁₋₄alkyl, C₁₋₄alkylene-vinyl, ethynyl, ethynylC₁₋₄alkyl, C₁₋₄alkylene-ethynyl, are optionally substituted with a C₃₋₆cycloakyl or a benzene ring, wherein the benzene ring is optionally substituted with a halogen, OH or CH₃.

6. The compound or a pharmaceutically acceptable salt or prodrug thereof according to claim 1, wherein R¹ is selected from:

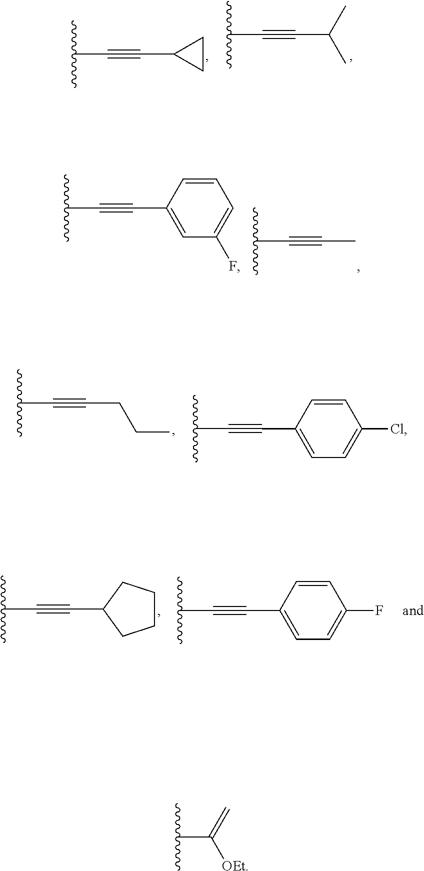

7. A pharmaceutical composition comprising:
i. a compound according to claim 1, or a pharmaceutically acceptable sat, and
ii. at least one pharmaceutical acceptable carrier.

8. A method of treating or preventing a viral infection in a subject, the method comprising administering to the subject the pharmaceutical composition according to claim 1.

9. A method of treating or preventing a viral infection in a subject, the method comprising administering to the subject the pharmaceutical composition according to claim 7.

10. The method of claim 8, wherein the viral infection is an infection by an RNA virus.

11. The method of claim 10, wherein the RNA virus is selected from coronavirus, measles, tacaribe virus, yellow fever virus, influenza virus, Chikungunya, dengue, respiratory syncytial virus (RSV), human immunodeficiency virus (HIV) and norovirus.

12. The method of claim 9, wherein the viral infection is an infection by an RNA virus.

13. The method of claim 12, wherein the RNA virus is selected from coronavirus, measles, tacaribe virus, yellow fever virus, influenza virus, Chikungunya, dengue, respiratory syncytial virus (RSV), human immunodeficiency virus (HIV) and norovirus.
